(19) United States
(12) Patent Application Publication
(10) Pub. No.: US 2004/0115727 A1
(43) Pub. Date: Jun. 17, 2004

(54) Evolved clostridial toxins with altered protease specificity

(75) Inventors: Lance E. Steward, Irvine, CA (US); Kei Roger Aoki, Coto de Caza, CA (US)

Correspondence Address: CAMPBELL & FLORES LLP, 4370 LA JOLLA VILLAGE DRIVE, 7TH FLOOR, SAN DIEGO, CA, 92122, US

(73) Assignee: Allergan, Inc., a Corporation

(21) Appl. No.: 10/318,417

(22) Filed: Dec. 11, 2002

(51) Int. Cl. G01N 33/53; C12N 15/09

(52) U.S. Cl. 435/7.1

(57) ABSTRACT

The present invention provides a method of producing an evolved clostridial toxin light chain having altered protease specificity by (a) generating a population, each member of which contains a clostridial toxin light chain variant or functional fragment thereof; (b) assaying the population for protease activity towards a selected clostridial toxin-resistant target protein, where increased protease activity is indicative of an evolved clostridial toxin light chain; and (c) isolating from the population one or more members, which contain an evolved clostridial toxin light chain or functional fragment thereof. Also provided herein are compositions which contain an evolved clostridial toxin light chain or functional fragment thereof having altered protease specificity.

### BACKGROUND OF THE INVENTION

1. Field of the Invention

The present invention relates generally to directed evolution and clostridial toxins and, more specifically, to evolved clostridial toxin light chains with altered protease specificity.

2. Background Information

Clostridial neurotoxins are highly potent and specific poisons of neural cells, with the human lethal dose of the botulinum toxins on the order of nanograms. However, in spite of their potentially deleterious effects, low controlled doses of botulinum neurotoxins have been successfully used as therapeutics.

The seven immunologically distinct botulinum neurotoxin serotypes (BoNT/A, BoNT/B, BoNT/C₁, BoNT/D, BoNT/E, BoNT/F and BoNT/G) exhibit several conserved functionalities: a binding domain responsible for targeting the toxin to the nerve terminus; a translocation domain that facilitates translocation across the endosomal membrane; and a zinc-metalloprotease domain. A “heavy chain” encodes the first two of these functions while a “light chain” contains the protease domain. The different botulinum serotypes also share the same fundamental mechanism of action involving inhibition of acetylcholine release at the synaptic junction following zinc-metalloprotease cleavage of a SNARE target protein. Each neurotoxin serotype cleaves a distinct site present within one of three neurotoxin-sensitive SNARE proteins: vesicle associated protein (VAMP), SNAP-25 or syntaxin.

Thus, the specificity of naturally occurring botulinum neurotoxins is restricted to a limited number of neurotoxin-sensitive SNARE proteins. Neurotoxin proteases with novel proteolytic activity for a neurotoxin-resistant SNARE protein or for another target protein of therapeutic interest, such as an over-expressed or poorly cleared protein that contributes to disease, are presently not available. Thus, there is a need for evolved clostridial toxin light chains having altered protease specificity, which can be used, for example, as novel therapeutics. The present invention satisfies this need and provided related advantages as well.

### SUMMARY OF THE INVENTION

The present invention provides a method of producing an evolved clostridial toxin light chain having altered protease specificity by (a) generating a population, each member of which contains a clostridial toxin light chain variant or functional fragment thereof; (b) assaying the population for protease activity towards a selected clostridial toxin-resistant target protein, where increased protease activity is indicative of an evolved clostridial toxin light chain; and (c) isolating from the population one or more members, which contain an evolved clostridial toxin light chain or functional fragment thereof. In a method of the invention, the altered protease specificity can be, for example, for a clostridial toxin-resistant SNARE protein such as human SNAP-23, syncollin or TI-VAMP. The clostridial toxin light chain variants can be, for example, botulinum toxin light chain variants such as BoNT/A, BoNT/B, BoNT/C₁, BoNT/D, BoNT/E, BoNT/F or BoNT/G light chain variants or tetanus toxin (TeNT) light chain variants.

A variety of populations can be assayed according to a method of the invention including, without limitation, random populations. In one embodiment of the invention, a population of clostridial toxin light chain variants or functional fragments thereof is produced by expressing a population of nucleic acid molecules. Genetic modification of one or more nucleic acid molecules encoding a clostridial toxin light chain or segment thereof can be useful in producing such a population of nucleic acid molecules. Genetic modifications useful in the invention include, but are not limited to, random mutagenesis, which can be used to produce, for example, a population having at least 10² different members each containing a clostridial toxin light chain variant or functional fragment thereof, or a population having at least 10³ different members each containing a clostridial toxin light chain variant or functional fragment thereof.

In one embodiment, random mutagenesis of one or more nucleic acid molecules is performed to yield an average of 1 to 3 amino acid substitutions per clostridial toxin light chain variant or functional fragment thereof. As non-limiting examples, random mutagenesis can be performed using error-prone polymerase chain reaction amplification; DNA shuffling between two or more nucleic acid molecules encoding clostridial toxin light chains or segments thereof; or saturation mutagenesis of one or more codons of one or more nucleic acid molecules encoding clostridial toxin light chains or segments thereof.

A variety of means can be useful for assaying a population for protease activity towards a selected clostridial toxin-resistant target protein according to a method of the invention. In one embodiment, the invention is practiced by assaying a population of phage, each phage expressing a clostridial toxin light chain variant or functional fragment thereof. In another embodiment, the invention is practiced by assaying a population of microorganisms which each express a clostridial toxin light chain variant or functional fragment thereof. In a further embodiment, the invention is practiced by assaying a population of microorganisms which each express on the cell surface a clostridial toxin light chain variant or functional fragment thereof. Microorganisms useful in the invention encompass, without limitation, bacteria such as Escherichia coli. In yet another embodiment, the invention is practiced by selecting from the population one or more viable members, which each contain an evolved clostridial toxin light chain or functional fragment thereof.

The invention also can be practiced by assaying a population of purified or partially purified polypeptides, or functional fragments thereof, for protease activity towards a selected clostridial toxin-resistant target protein. Such a population can be, for example, a population of purified clostridial toxin light chain variants or functional fragments thereof. Such a population also can be a population of purified toxins, which contain a clostridial toxin heavy chain and a clostridial toxin light chain variant. In one embodiment, the invention is practiced by assaying a population of purified dichain toxins containing clostridial toxin light chain variants.

A variety of techniques can be useful in assaying for protease activity towards a selected clostridial toxin-resistant target protein. Such techniques include, yet are not limited to, immunoassays such as enzyme-linked immunosorbent assays, fluorescence resonance energy transfer assays, and fluorescence activated cell sorting assays. In one embodiment, the steps of the invention are repeated one or more times. In another embodiment, the steps of the invention are repeated three or more times.

The present invention also provides a composition which contains an evolved clostridial toxin light chain or functional fragment thereof having altered protease specificity. The altered protease specificity can be, for example, for a clostridial toxin-resistant SNARE protein such as, without limitation, human SNAP-23. In one embodiment, the invention provides a composition containing an evolved clostridial toxin light chain or functional fragment thereof having altered protease specificity, where, under the appropriate conditions, the altered protease specificity inhibits exocytosis. In another embodiment, the invention provides a composition containing an evolved clostridial toxin light chain or functional fragment thereof having altered protease specificity, where, under the appropriate conditions, the altered protease specificity inhibits neuronal exocytosis. In a further embodiment, the invention provides a composition containing an evolved clostridial toxin light chain or functional fragment thereof having altered protease specificity, where, under the appropriate conditions, the altered protease specificity inhibits secretory cell exocytosis such as pancreatic acinar cell exocytosis.

An evolved clostridial toxin light chain or functional fragment thereof can differ from a naturally occurring clostridial toxin light chain by, for example, one or more amino acid substitutions. In one embodiment, the evolved clostridial toxin light chain or functional fragment thereof differs from a naturally occurring clostridial toxin light chain by at most three amino acid substitutions. In another embodiment, the evolved clostridial toxin light chain or functional fragment thereof differs from a naturally occurring clostridial toxin light chain by a single amino acid substitution.

A composition of the invention optionally includes a clostridial toxin heavy chain. In one embodiment, a composition of the invention includes a clostridial toxin heavy chain which has a non-naturally occurring amino acid sequence. In another embodiment, a composition of the invention includes a clostridial toxin heavy chain which has a non-naturally occurring binding domain. It is understood that the compositions of the invention encompass evolved single-chain and dichain toxins.

The present invention also provides a nucleic acid molecule containing a nucleic acid sequence that encodes an evolved clostridial toxin light chain having altered protease specificity, or a functional fragment thereof. The altered protease specificity can be, for example, for a clostridial toxin-resistant SNARE protein such as human SNAP-23. In one embodiment, the encoded evolved clostridial toxin light chain or functional fragment thereof has altered protease specificity, which, under the appropriate conditions, inhibits exocytosis. In another embodiment, the encoded evolved clostridial toxin light chain or functional fragment thereof has altered protease specificity, which, under the appropriate conditions, inhibits neuronal exocytosis. In a further embodiment, the encoded evolved clostridial toxin light chain or functional fragment thereof has altered protease specificity, which, under the appropriate conditions, inhibits secretory cell exocytosis such as pancreatic acinar cell exocytosis.

In a nucleic acid composition of the invention, the encoded evolved clostridial toxin light chain can differ from a naturally occurring clostridial toxin light chain by one or more amino acid substitutions, and, in one embodiment, differs from a naturally occurring clostridial toxin light chain by at most three amino acid substitutions. In another embodiment, the encoded evolved clostridial toxin light chain differs from a naturally occurring clostridial toxin light chain by a single amino acid substitution. A nucleic acid molecule of the invention can optionally include a nucleic acid sequence encoding a clostridial toxin heavy chain.

### BRIEF DESCRIPTION OF THE DRAWINGS

FIG. 1 shows a schematic of the deduced structure and postulated mechanism of activation of clostridial neurotoxins. Toxins can be produced as an inactive single polypeptide chain of 150 kDa, composed of three 50 kDa domains connected by loops. Selective proteolytic cleavage activates the toxins by generating two disulfide-linked chains: the light (L) chain of 50 kDa and the heavy (H) chain of 100 kDa, which is made up of two domains denoted H_{N} and H_{C}. The three domains play distinct roles: the C-terminal domain of the heavy chain (H_{C}) functions in cell binding while the N-terminal domain of the heavy chain (H_{N}) permits translocation from endosome to cell cytoplasm. Following reduction of the disulfide linkage inside the cell, the zinc-endopeptidase activity of the light chain is liberated.

FIG. 2 shows a schematic of the four steps required for tetanus and botulinum toxin activity in central and peripheral neurons.

FIG. 3 shows the subcellular localization at the plasma membrane and sites of cleavage of SNAP-25, VAMP and syntaxin. VAMP is bound to synaptic vesicle membrane, whereas SNAP-25 and syntaxin are bound to the target plasma membrane. BoNT/A and /E cleave SNAP-25 close to the carboxy-terminus, releasing nine or 26 residues, respectively. BoNT/B, /D, /F, /G and TeNT act on the conserved central portion of VAMP (dotted) and release the amino-terminal portion of VAMP into the cytosol. BoNT/C1 cleaves SNAP-25 close to the carboxy-terminus as well as cleaving syntaxin at a single site near the cytosolic membrane surface. The action of BoNT/B, /C1, /D, /F, /G and TeNT results in release of a large portion of the cytosolic domain of VAMP or syntaxin, while only a small portion of SNAP-25 is released by selective proteolysis by BoNT/A, /C1 or /E.

FIG. 4 shows the neurotoxin recognition motif of VAMP, SNAP-25 and syntaxin. (A) Hatched boxes indicate the presence and positions of a motif common to the three targets of clostridial neurotoxins. (B) The recognition motif is composed of hydrophobic residues (“h”); negatively charged Asp or Glu residues (“−”) and polar residues (“p”); “x” represents any amino acid. The motif is included in regions of VAMP, SNAP-25 and syntaxin predicted to adopt an α-helical conformation. (C) A top view of the motif in an α-helical conformation is shown. Negatively charged residues align on one face, while hydrophobic residues align on a second face.

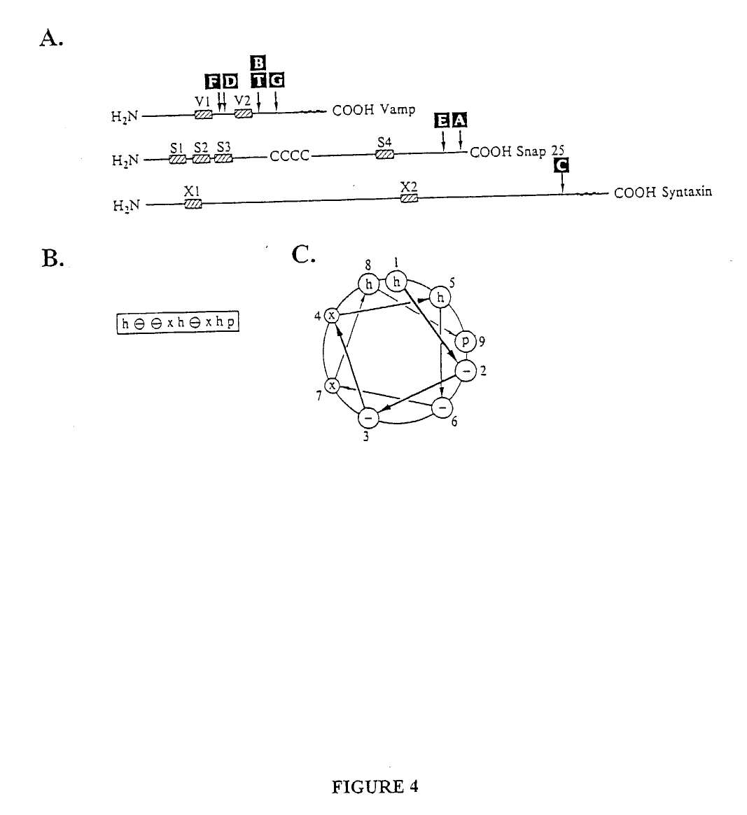

FIG. 5 shows a sequence alignment of human SNAP-23a (SEQ ID NO: 1), human SNAP-23b (SEQ ID NO: 2) and human SNAP-25 (SEQ ID NO: 3). The BoNT/A and BoNT/E cleavage sites are indicated by a vertical line. The minimum region required for binding of SNAP-25 by BoNT/A is boxed in gray, and the minimum region required for binding of SNAP-25 by BoNT/E is boxed in white.

### DETAILED DESCRIPTION OF THE INVENTION

The tetanus and botulinum neurotoxins to which the invention relates, together denoted “clostridial” toxins, cause the neuroparalytic syndromes of tetanus and botulism, with tetanus toxin acting mainly within the central nervous system and botulinum toxin acting on the peripheral nervous system. Clostridial neurotoxins share a similar mechanism of cell intoxication in which the release of neurotransmitters is blocked. In these toxins, which are composed of two disulfide-linked polypeptide chains, the larger subunit (“heavy chain”) is responsible for neurospecific binding and translocation of the smaller subunit into the cytoplasm. Upon translocation and reduction in neurons, the smaller chain (“light chain”) displays protease activity specific for protein components involved in neuroexocytosis. The “SNARE” protein targets of clostridial toxins are common to exocytosis in a variety of non-neuronal cell types; in these cells, as in neurons, light chain protease activity inhibits exocytosis.

Distinct SNARE sequences in the SNARE target proteins VAMP, SNAP-25 and syntaxin are recognized by different clostridial toxins. Tetanus neurotoxin and botulinum neurotoxins B, D, F, and G specifically recognize VAMP (also known as synaptobrevin), an integral protein of the synaptic vesicle membrane. VAMP is cleaved at distinct bonds depending on the neurotoxin. Botulinum A and E neurotoxins recognize and specifically cleave SNAP-25, a protein of the presynaptic membrane, at two different sites in the carboxy-terminal portion of the protein. Botulinum neurotoxin C cleaves syntaxin, a protein of the nerve plasmalemma, in addition to SNAP-25. The three protein targets of the clostridial neurotoxins are conserved from yeast to humans, although cleavage and toxin susceptibility are not necessarily conserved in all species (see Humeau et al., Biochimie 82:427-446 (2000); Niemann et al., Trends in Cell Biol. 4:179-185 (1994); and Pellizzari et al., Phil. Trans. R. Soc. London 354:259-268 (1999)).

Naturally occurring tetanus and botulinum neurotoxins are produced as inactive polypeptide chains of 150 kDa without a leader sequence. These toxins may be cleaved by bacterial or tissue proteinases at an exposed protease-sensitive loop, generating active dichain toxin. Naturally occurring clostridial toxins contain a single interchain disulfide bond bridging the heavy chain (H, 100 kDa) and light chain (L, 50 kDa); such a bridge is important for neurotoxicity of toxin added extracellularly (Montecucco and Schiavo, Quarterly Rev. Biophysics 28:423-472 (1995)).

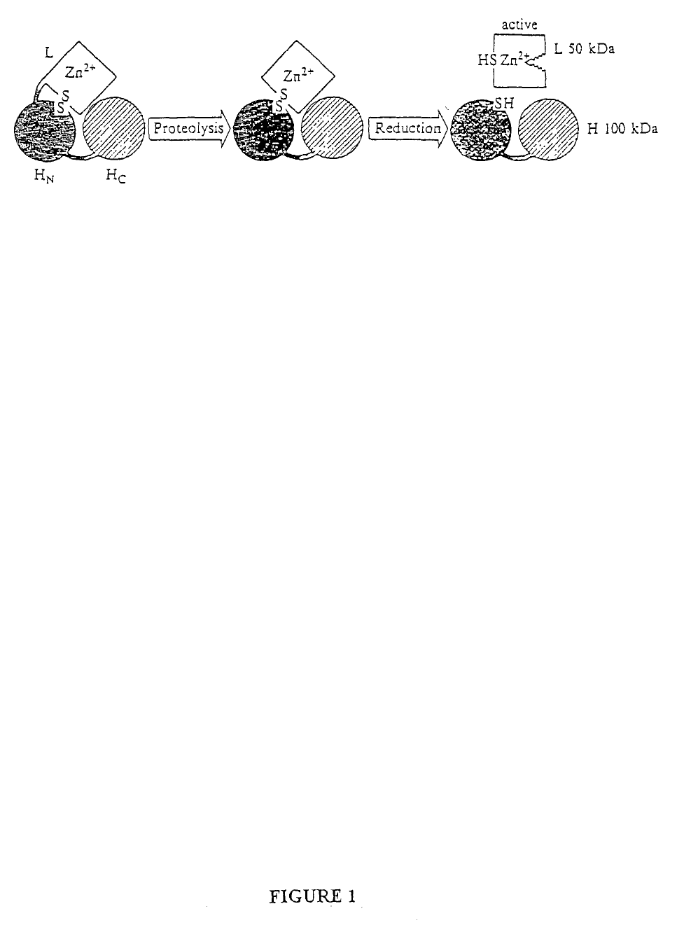

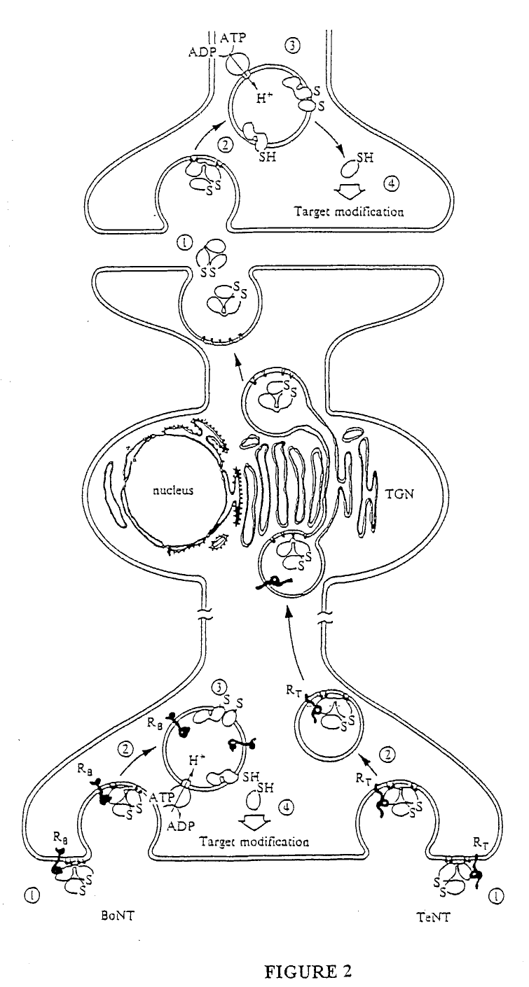

The clostridial toxins appear to be folded into three distinct 50 kDa domains, as shown in FIG. 1, with each domain having a distinct functional role. As illustrated in FIG. 2, the cell intoxication mechanism of the clostridial toxins consists of four distinct steps: (1) binding; (2) internalization; (3) membrane translocation; and (4) enzymatic cleavage of target protein. The carboxy-terminal portion of the heavy chain (H_{C}) functions in neurospecific binding, while the amino-terminal portion of the heavy chain (H_{N}) functions in membrane translocation. The light chain is responsible for the intracellular catalytic activity as discussed further below (Montecucco and Schiavo, supra, 1995).

The amino acid sequences of eight human clostridial neurotoxins have been derived from the corresponding genes, and comparison of the nucleotide and amino acid sequences indicates that the clostridial toxins derive from a common ancestral gene (Neimann, “Molecular Biology of Clostridial Neurotoxins” in Sourcebook of Bacterial Protein Toxins Alouf and Freer (Eds.) pp. 303-348 London: Academic Press 1991). Sequence variations among the seven botulinum toxins also have been observed (Humeau et al., supra, 2000). The light and heavy chains are composed of roughly 439 and 843 residues, respectively. Homologous segments are separated by regions of little or no similarity. The most well conserved regions of the light chain among the various toxin serotypes are the amino-terminal region of about 100 residues and the central region corresponding to residues 216 to 244 of TeNT, as well as the two cysteines forming the interchain disulfide bond. The 216 to 244 region contains a His-Glu-X-X-His binding motif characteristic of zinc-metalloproteases. The clostridial toxin heavy chains are less well conserved than the light chains, with the carboxy-terminal portion of H_{C} corresponding to residues 1140 to 1315 of TeNT being the most variable. This is consistent with the involvement of the H_{C} domain in binding to nerve terminals and the fact that different neurotoxin serotypes appear to bind different receptors.

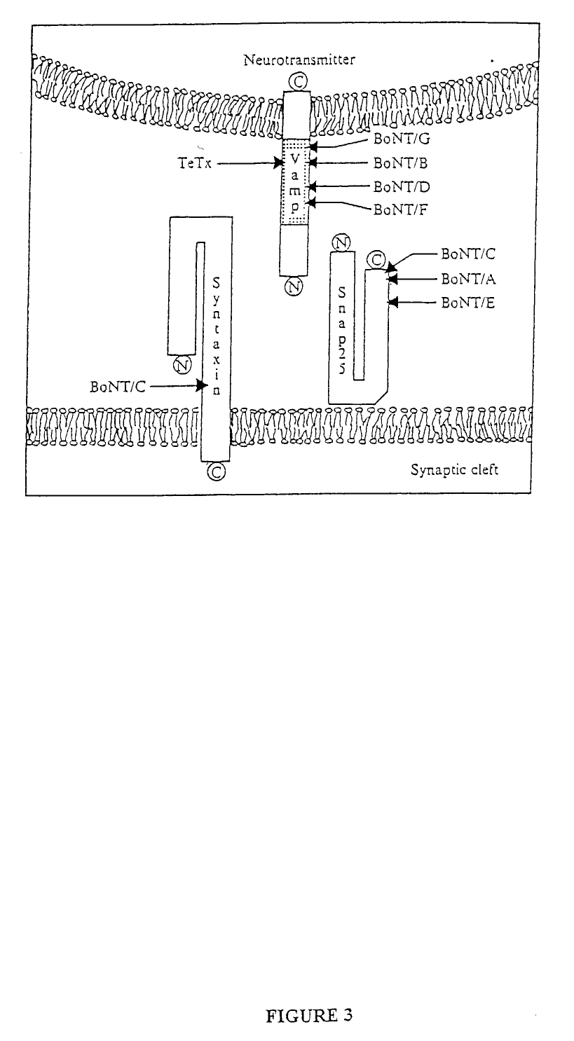

As discussed above, natural targets of the clostridial neurotoxins include VAMP, SNAP-25, and syntaxin. VAMP is bound to the synaptic vesicle membrane, whereas SNAP-25 and syntaxin are bound to the target membrane (see FIG. 3). BoNT/A and BoNT/E cleave SNAP-25 in the carboxy-terminal region, releasing nine or twenty-six amino acid residues, respectively, and BoNT/C1 also cleaves SNAP-25 near the carboxy-terminus. The botulinum serotypes BoNT/B, BoNT/D, BoNT/F and BoNT/G, and tetanus toxin, act on the conserved central portion of VAMP, and release the amino-terminal portion of VAMP into the cytosol. BoNT/C1 cleaves syntaxin at a single site near the cytosolic membrane surface. Thus, proteolytic cleavage by BoNT/B, BoNT/C1, BoNT/D, BoNT/F, BoNT/G or TeNT releases of a large portion of the cytosolic domain of VAMP or syntaxin, while only a small portion of SNAP-25 is released by BoNT/A, BoNT/C1 or BoNT/E cleavage (FIG. 3; see, also, Montecucco and Schiavo, supra, 1995).

Naturally occurring VAMP is a protein of about 120 residues, with the exact length depending on the species and isotype. As shown in FIG. 3, VAMP contains a short carboxy-terminal segment inside the vesicle lumen, with the majority of the molecule exposed to the cytosol. Although the proline-rich amino-terminal thirty residues are divergent among species and isoforms, the central portion of VAMP (residues 30 to 96), which is rich in charged and hydrophilic residues and includes known cleavage sites, is highly conserved. VAMP colocalizes with synaptophysin on the synaptic vesicle membrane.

A variety of species homologs of VAMP are known in the art including human, rat, bovine, Torpedo, Drosophila, yeast, squid and Aplysia homologs. In addition, multiple isoforms of VAMP have been identified including VAMP-1, VAMP-2 and cellubrevin, and toxin-insensitive forms have been identified in non-neuronal cells. VAMP appears to be present in all vertebrate tissues although the distribution of VAMP-1 and VAMP-2 varies in different cell types. Chicken and rat VAMP-1 are not cleaved by TeNT or BoNT/B. These VAMP-1 homologs have a valine in place of the glutamine present in human and mouse VAMP-1 at the TeNT or BoNT/B cleavage site. The substitution does not effect BoNT/D, /F or /G, which cleave both VAMP-1 and VAMP-2 with similar rates.

Naturally occurring SNAP-25, a protein of about 206 residues lacking a transmembrane segment, is associated with the cytosolic surface of the nerve plasmalemma (FIG. 3; see, also, Hodel et al., Int. J. Biochemistry and Cell Biology 30:1069-1073 (1998)). In addition to homologs highly conserved from Drosophila to mammals, SNAP-25-related proteins have been cloned from yeast. SNAP-25 is required for axonal growth during development and may be required for nerve terminal plasticity in the mature nervous system. In humans, two isoforms are differentially expressed during development; SNAP-25a is constitutively expressed during fetal development, while SNAP-25b appears at birth and predominates in adult life. SNAP-25 analogs such as the toxin-resistant analog, SNAP-23, also are expressed outside the nervous system, for example, in pancreatic cells (Ravichandran et al., J. Biol. Chem. 271:13300-13303 (1996); Mollinedo and Lazo, Biochem. Biophys. Res. Comm. 231:808-812 (1997); Macaulay et al. Biochem. Biophys. Res. Comm. 237:388-393 (1997); and Chen et al., Biochem. 36:5719-5728 (1997)).

Syntaxin is located on the cytosolic surface of the nerve plasmalemma and is membrane-anchored via a carboxy-terminal segment, with most of the protein exposed to the cytosol. Syntaxin colocalizes with calcium channels at the active zones of the presynaptic membrane, where neurotransmitter release takes place. In addition, syntaxin interacts with synaptotagmin, a protein of the SSV membrane, that forms a functional bridge between the plasmalemma and vesicles. A variety of syntaxin isoforms have been identified. Two isoforms of slightly different length (285 and 288 residues) have been identified in nerve cells (isoforms 1A and 1B), and isoforms 2, 3, 4 and 5 are expressed in other tissues. The different isoforms have varying sensitivities to BoNT/C1, with the 1A, 1B, 2 and 3 syntaxin isoforms cleaved by this toxin, and isoforms 4 and 5 resistant to cleavage.

The naturally occurring, non-evolved clostridial toxins cleave specific and distinct cleavage sites. In standard nomenclature, the sequence surrounding a clostridial toxin cleavage site is denoted P₅-P₄-P₃-P₂-P₁-P₁′-P₂′-P₃′-P₄′-P₅′, with P₁-P₁′ representing the scissile bond. As shown in Table 1, naturally occurring BoNT/A cleaves a Gln-Arg bond; naturally occurring BoNT/B and TeNT cleave a Gln-Phe bond; naturally occurring BoNT/C1 cleaves a Lys-Ala or Arg-Ala bond; naturally occurring BoNT/D cleaves a Lys-Leu bond; naturally occurring BoNT/E cleaves an Arg-Ile bond; naturally occurring BoNT/F cleaves a Gln-Lys bond; and naturally occurring BoNT/G cleaves an Ala-Ala bond.

In contrast to naturally occurring clostridial toxins, the invention provides “evolved clostridial toxins,” which have non-naturally occurring protease specificities. Thus, the present invention provides a composition which contains an evolved clostridial toxin light chain or functional fragment thereof having altered protease specificity. The altered protease specificity can be, for example, for a clostridial toxin-resistant SNARE protein such as, without limitation, human SNAP-23. In one embodiment, the invention provides a composition containing an evolved clostridial toxin light chain or functional fragment thereof having altered protease specificity, where, under the appropriate conditions, the altered protease specificity inhibits exocytosis. In another embodiment, the invention provides a composition containing an evolved clostridial toxin light chain or functional fragment thereof having altered protease specificity, where, under the appropriate conditions, the altered protease specificity inhibits neuronal exocytosis. In a further embodiment, the invention provides a composition containing an evolved clostridial toxin light chain or functional fragment thereof having altered protease specificity, where, under the appropriate conditions, the altered protease specificity inhibits secretory cell exocytosis such as pancreatic acinar cell exocytosis.

TABLE 1
BONDS CLEAVED IN HUMAN VAMP-2, SNAP-25 OR
SYNTAXIN BY NATURALLY OCCURRING
CLOSTRIDIAL TOXINS

| | | |
|---|---|---|
| Toxin | Target | P₄-P₃-P₂-P₁ -- P₁′-P₂′-P₃′-P₄′ |
| BoNT/A | SNAP-25 | Glu-Ala-Asn-Gln-Arg*-Ala-Thr-Lys |
| | | SEQ ID NO: 4 |
| BoNT/B | VAMP-2 | Gly-Ala-Ser-Gln-Phe*-Glu-Thr-Ser |
| | | SEQ ID NO: 5 |
| BoNT/C1 | syntaxin | Asp-Thr-Lys-Lys-Ala*-Val-Lys-Tyr |
| | | SEQ ID NO: 6 |
| BoNT/D | VAMP-2 | Arg-Asp-Gln-Lys-Leu*-Ser-Glu-Leu |
| | | SEQ ID NO: 7 |
| BoNT/E | SNAP-25 | Gln-Ile-Asp-Arg-Ile*-Met-Glu-Lys |
| | | SEQ ID NO: 8 |
| BoNT/F | VAMP-2 | Glu-Arg-Asp-Gln-Lys*-Leu-Ser-Glu |
| | | SEQ ID NO: 9 |
| BoNT/G | VAMP-2 | Glu-Thr-Ser-Ala-Ala*-Lys-Leu-Lys |
| | | SEQ ID NO: 10 |
| TeNT | VAMP-2 | Gly-Ala-Ser-Gln-Phe*-Glu-Thr-Ser |
| | | SEQ ID NO: 11 |

Scissile bond shown in bold

An evolved clostridial toxin light chain or functional fragment thereof can differ from a naturally occurring clostridial toxin light chain by, for example, one or more amino acid substitutions. In one embodiment, the evolved clostridial toxin light chain or functional fragment thereof differs from a naturally occurring clostridial toxin light chain by at most three amino acid substitutions. In another embodiment, the evolved clostridial toxin light chain or functional fragment thereof differs from a naturally occurring clostridial toxin light chain by a single amino acid substitution. In a further embodiment, an evolved clostridial toxin light chain or functional fragment of the invention forms the maximal number of hydrogen bonds with residues in the selected clostridial toxin-resistant target protein which have the potential to hydrogen bond, such as Ser, Thr, Tyr, Asp, Glu, Asn or Gln. In yet a further embodiment, the evolved clostridial toxin light chain forms at least as many hydrogen bonds with the selected clostridial toxin-resistant target protein as the naturally occurring clostridial toxin light chain forms with its naturally occurring SNARE target protein.

A composition of the invention optionally includes a clostridial toxin heavy chain. In one embodiment, a composition of the invention includes a clostridial toxin heavy chain which has a non-naturally occurring amino acid sequence. In another embodiment, a composition of the invention includes a clostridial toxin heavy chain which has a non-naturally occurring binding domain.

The present invention also provides a nucleic acid molecule containing a nucleic acid sequence that encodes an evolved clostridial toxin light chain having altered protease specificity, or a functional fragment thereof. The altered protease specificity can be, for example, for a clostridial toxin-resistant SNARE protein such as human SNAP-23. In one embodiment, the encoded evolved clostridial toxin light chain or functional fragment thereof has altered protease specificity, which, under the appropriate conditions, inhibits exocytosis. In another embodiment, the encoded evolved clostridial toxin light chain or functional fragment thereof has altered protease specificity, which, under the appropriate conditions, inhibits neuronal exocytosis. In a further embodiment, the encoded evolved clostridial toxin light chain or functional fragment thereof has altered protease specificity, which, under the appropriate conditions, inhibits secretory cell exocytosis such as pancreatic acinar cell exocytosis.

In a nucleic acid composition of the invention, the encoded evolved clostridial toxin light chain can differ from a naturally occurring clostridial toxin light chain by one or more amino acid substitutions, and, in one embodiment, differs from a naturally occurring clostridial toxin light chain by at most three amino acid substitutions. In another embodiment, the encoded evolved clostridial toxin light chain differs from a naturally occurring clostridial toxin light chain by a single amino acid substitution. A nucleic acid molecule of the invention can optionally include a nucleic acid sequence encoding a clostridial toxin heavy chain.

An evolved clostridial toxin light chain of the invention is a clostridial toxin light chain which has a non-naturally occurring amino acid sequence and altered protease specificity as compared to naturally occurring clostridial toxins. As used herein, the term “altered protease specificity” means that the amino acid sequence recognized by the evolved clostridial toxin is distinct from amino acid sequences recognized by naturally occurring clostridial toxins. It is understood that altered protease specificity encompasses proteolysis of cleavage sites which are distinct from cleavage sites cleaved by naturally occurring clostridial toxins and also encompasses proteolysis of cleavage sites identical to the sites cleaved by naturally occurring clostridial toxins, where the surrounding or adjacent recognition sequence is distinct from the recognition sequences of naturally occurring clostridial toxins. In one embodiment, the evolved clostridial toxin light chain has protease specificity for a scissile bond which is identical to the scissile bond cleaved by a naturally occurring clostridial toxin light chain.

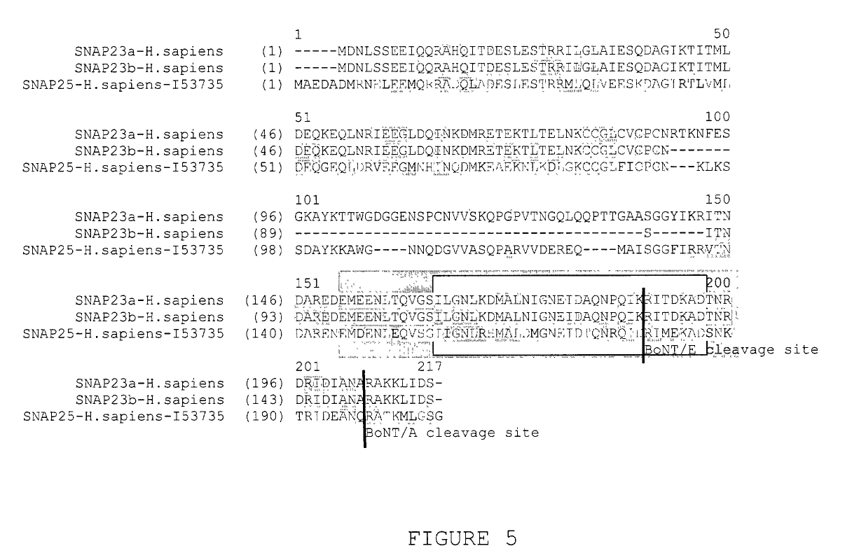

As shown in FIG. 5, SNAP-23a and SNAP-23b are toxin-resistant proteins which differ from SNAP-25 at several residues within the minimum region required for binding of BoNT/E or BoNT/A, and also differ at the sites corresponding to BoNT/E or BoNT/A cleavage sites. A clostridial toxin light chain variant which cleaves SNAP-23a or SNAP-23b is one example of an evolved clostridial toxin having “altered protease specificity” as defined herein.

An evolved clostridial toxin light chain of the invention can cleave a selected clostridial toxin-resistant target protein. As used herein, the term “clostridial toxin-resistant target protein” means a protein that is not detectably cleaved by naturally occurring clostridial toxins under conditions suitable for clostridial toxin protease activity. A “selected” clostridial toxin-resistant target protein is a clostridial toxin-resistant target protein of interest. Clostridial toxin-resistant target proteins include proteins specifically or non-specifically expressed in motor or sensory neurons, as well as proteins expressed in non-neuronal cells including, without limitation, secretory cells such as pancreatic acinar cells.

As a non-limiting example, a clostridial toxin-resistant target protein can be a clostridial toxin-resistant SNARE protein. As used herein, the term “SNARE protein” is synonymous with “soluble N-ethylmaleimide-sensitive factor attachment protein receptor” and means a cytoplasmically oriented membrane-associated protein that facilitates membrane fusion. The term SNARE protein encompasses SNAREs located on transport vesicles (v-SNAREs) as well as SNAREs located on the surface of secretory organelles (t-SNAREs) and further encompasses SNAREs involved in apical as well as basolateral exocytosis (see, for example, Gerst, Cellular and Molecular Life Sciences 55:707-734 (1999); and Banfield, Trends in Biochem. Sci. 26:67-68 (2001)). The term SNARE further encompasses Q- and R-SNAREs, which include a conserved glutamine or arginine, respectively, within the SNARE-binding domain.

SNARE proteins encompass, yet are not limited to, a variety of isoforms and species homologs of VAMP, SNAP-25, SNAP-23, synaptotagmin and syntaxin. As discussed above, in nature, several SNAREs are sensitive to cleavage by one or more clostridial toxins, while others are resistant to cleavage. As used herein, the term “clostridial toxin-resistant SNARE protein” means a SNARE protein that is not detectably cleaved by naturally occurring clostridial toxins under conditions suitable for clostridial toxin protease activity. Clostridial toxin-resistant SNARE proteins encompass resistant forms of SNARE proteins normally cleaved by a toxin such as resistant forms of SNAP-25, VAMP or syntaxin. A clostridial toxin-resistant SNARE protein can be naturally expressed in neuronal cells, including sensory or motor neurons or both, and further can be selectively or specifically expressed in neuronal cells, for example, with little or no expression in other cell types. It is understood that a clostridial toxin-resistant SNARE protein also can be expressed in non-neuronal cells such as, without limitation, secretory cells; pancreatic acinar cells; inner medullary collecting duct (IMCD) cells of the kidney; platelets; neutrophils; eosinophils; lymphocytes; phagocytes; mast cells; epithelial cells, for example, on the apical plasma membrane; adipocytes and muscle cells.

Clostridial toxin-resistant target proteins also encompass a variety of proteins other than SNAREs such as proteins which accumulate due to overexpression or poor clearance and which are associated with disease. Clostridial toxin-resistant target proteins include, without limitation, multidrug resistance proteins and proteins that, upon cleavage, trigger an apoptotic pathway. In one embodiment, the clostridial toxin-resistant target protein is associated with cancer. In another embodiment, the clostridial toxin-resistant target protein is associated with a neurological or neurodegenerative disorder such as Huntington's disease, Alzheimer's disease or Parkinson's disease. In a further embodiment, the clostridial toxin-resistant target protein is associated with an immune-mediated disorder such as allergy or asthma. In another embodiment, the clostridial toxin-resistant target protein is associated with an autoimmune disorder such as multiple sclerosis. In yet another embodiment, the clostridial toxin-resistant target protein is a PrP Sc protein associated with a prion disease such as Creutzfeld-Jakob Disease (CJD), scrappie or bovine spongiform encelphalopathy.

In some cases, a clostridial toxin-resistant target protein has a mutated amino acid sequence that differs at one or more amino acid positions from the corresponding wild type protein, as in the case of ras. It is understood that an evolved clostridial toxin light chain of the invention can be evolved to specifically cleave a mutated target protein, without cleaving the corresponding wild type protein, or can be evolved to cleave both mutant and wild type forms of a protein. As one example, an evolved clostridial toxin light chain of the invention can have protease specificity for the activated form of ras (v-ras), while lacking protease specificity for wild type ras (c-ras).

A variety of oncogenic proteins, or proteins that promote cell survival, can contribute to cancer and can be a clostridial toxin-resistant target protein cleaved by an evolved clostridial toxin light chain as defined herein. Such proteins include, without limitation, Bcl-2, Bcl-X_{L} and other anti-apoptotic Bcl-2 family members; members of the inhibitor of apoptosis (IAP) family such as c-IAP-1, c-IAP-2, XIAP and NIAP; protein kinase C; Ha-ras; c-Raf-1; c-Myc; c-Myb; DNA methyltransferase; ribonucleotide reductase; and tumor type-specific proteins such as the BR-3 gene product specifically expressed in glioma (Orr and O'Neill, Curr. Opin. Mol. Ther. 2:325-331 (2000); Anderson, Trends Pharm. Sci. 18:51 (1997); Gross et al., Genes Dev. 13:1899-1911 (1999); Deveraux and Reed, Genes Dev. 13:239-252 (1999); and Weil et al., Anticancer Res. 22:1467-1474 (2002)). Thus, Bcl-2 or BCl-X_{L} or a related anti-apoptotic family member; c-IAP-1, c-IAP-2, XIAP or NIAP or another IAP family member; protein kinase C; Ha-ras; c-Raf-1; c-Myc; c-Myb; DNA methyltransferase; or ribonucleotide reductase can be a clostridial toxin-resistant target protein cleaved by an evolved clostridial toxin light chain of the invention. An evolved clostridial toxin light chain with protease activity towards such a cancer-associated target protein, or an encoding nucleic acid molecule, can therefore serve as an anti-cancer therapeutic.

A clostridial toxin-resistant target protein also can be a protein associated with a neurological disease. In particular embodiments, such a clostridial toxin-resistant target protein is associated with a neurodegenerative disorder such as, without limitation, Huntington's disease, Alzheimer's disease or Parkinson's disease. As a non-limiting example, caspase activation correlates with progression of Huntington's disease (Mejia and Friedlander, Neuroscientist 7:480-489 (2001)); thus, an evolved clostridial toxin light chain of the invention designed to proteolyze a caspase such as caspase-1 or caspase-3 can be a therapeutic agent useful for preventing or treating Huntington's disease. Similarly, tissue transglutaminase (tTG) can play a role in pathogenesis of Huntington's disease (Lesort et al., Neurochem. Int. 40:37052 (2002)), and proteolysis of tissue transglutaminase by an evolved clostridial toxin light chain of the invention can be used to prevent or treat Huntington's disease. Mutations of APP, presenilin 1 (PS1) or presenilin 2 (PS2) also can contribute to Alzheimer's disease as can expression of α-, β- or γ-secretases (Hardy and Hardy, Science 282:1075-1078 (1998); an evolved clostridial toxin light chain that proteolyzes a mutated form of APP, PS1 or PS2 or an α-, β- or γ-secretase can therefore be useful for preventing or treating Alzheimer's disease. In addition, an evolved clostridial toxin light chain that proteolyzes a P-Glycoprotein associated with drug-resistant epilepsy can be useful for treating this form of the disease (Rizzi et al., J. Neurosci. 22:5833-5839 (2002)).

Additional examples of clostridial toxin-resistant target proteins include the low-molecular-weight protein tyrosine phosphatase (LMPTP), which is associated with common diseases such as allergy, asthma, obesity, myocardial hypertrophy and Alzheimer's disease (Bottini et al., Arch. Immunol. Ther. Exp. (Warsz) 50:950194 (2002)); proteolysis of low-molecular-weight protein tyrosine phosphatase by an evolved clostridial toxin light chain can be used to treat or reduce susceptibility to these diseases. A clostridial toxin-resistant target protein also can be a protein that is selectively required for viability of phagocytes or lymphocytes; proteolysis of such a target protein by an evolved clostridial toxin light chain of the invention can be used to treat an autoimmune disease. A further example of a clostridial toxin-resistant target protein is the glucose type 4 transporter (GLUT4). One skilled in the art understands that an evolved clostridial toxin light chain of the invention can be designed to cleave any of these or other related or unrelated proteins including those which are mutated in a disease state or which are over-expressed or otherwise accumulate intracellularly in a disease state. Any such SNARE or non-SNARE protein is encompassed within the term clostridial toxin-resistant target protein as defined herein.

An evolved clostridial toxin light chain of the invention can be characterized, in part, by its “turnover number,” or k_{cat}, for a selected clostridial toxin-resistant target protein. k_{cat} is the rate of breakdown of the evolved light chain-substrate complex. An evolved clostridial toxin light chain can cleave a toxin-resistant target protein, for example, with a k_{cat} of about 0.001 to about 4000 sec⁻¹, or with a k_{cat} of about 1 to about 4000⁻¹. In particular embodiments, an evolved clostridial toxin light chain cleaves a toxin-resistant target protein with a k_{cat} of less than 1000 sec⁻, 500 sec⁻¹, 250 sec⁻¹, 100 sec⁻¹, 50 sec⁻¹, 20 sec⁻¹, 10 sec⁻¹, or 5 sec⁻¹. In further embodiments, an evolved clostridial toxin light chain cleaves a toxin-resistant target protein with a k_{cat} in the range of 1 to 1000 sec⁻¹; 1 to 500 sec⁻¹; 1 to 250 sec⁻¹; 1 to 100 sec⁻¹; 1 to 50 sec⁻¹; 10 to 1000 sec⁻¹; 10 to 500 sec⁻¹; 10 to 250 sec⁻¹; 10 to 100 sec⁻¹; 10 to 50 sec⁻¹; 25 to 1000 sec⁻¹; 25 to 500 sec⁻¹; 25 to 250 sec⁻¹; 25 to 100 sec⁻¹; 25 to 50 sec⁻¹; 50 to 1000 sec⁻¹; 50 to 500 sec⁻¹; 50 to 250 sec⁻¹; 50 to 100 sec⁻¹; 100 to 1000 sec⁻¹; 100 to 500 sec⁻¹; or 100 to 250 sec⁻¹. One skilled in the art understands the turnover number, k_{cat}, is assayed under standard kinetic conditions in which there is an excess of substrate. In still further embodiments, an evolved clostridial toxin light chain of the invention has a Michaelis constant (Km) for a selected clostridial toxin-resistant target protein of less than 1000 μM, less than 500 μM, less than 250 μM, less than 100 μM, less than 50 μM, less than 10 μM, less than 1 μM, less than 500 nM, less than 250 nM, less than 100 nM, less than 50 nM, less than 10 nM, less than 1 nM or less than 0.1 nM.

A composition of the invention can optionally include a clostridial toxin heavy chain; where included, the heavy chain can have a naturally occurring or non-naturally occurring amino acid sequence. Compositions of the invention having both an evolved clostridial toxin light chain and a heavy chain encompass, without limitation, single-chain and dichain toxins; single-chain pro-toxins which can be activated by cleavage at a heterologous protease site as described, for example, in WO/01 14570; compositions including a heavy chain having a naturally occurring sequence; compositions including a heavy chain having a non-naturally occurring sequence; compositions including a heavy chain with a non-naturally occurring binding domain, as described, for example, in U.S. Pat. No. 5,989,545; and compositions having a chimeric heavy chain, for example, those described in WO/00 61192. A composition of the invention further can contain an evolved clostridial toxin light chain or functional fragment together with a “transport protein,” such as one of those described, for example, in WO 95/32738.

Further provided herein is a method of producing an evolved clostridial toxin light chain having altered protease specificity by (a) generating a population, each member of which contains a clostridial toxin light chain variant or functional fragment thereof; (b) assaying the population for protease activity towards a selected clostridial toxin-resistant target protein, where increased protease activity is indicative of an evolved clostridial toxin light chain; and (c) isolating from the population one or more members, which contain an evolved clostridial toxin light chain or functional fragment thereof. In a method of the invention, the altered protease specificity can be, for example, for a clostridial toxin-resistant SNARE protein such as human SNAP-23, syncollin, TI-VAMP, syntaxin-3 or a resistant isoform or isotype of SNAP-25, VAMP or syntaxin (Galli et al., Mol. Biol. of the Cell 9:1437-1448 (1998)). The clostridial toxin light chain variants can be, for example, botulinum toxin light chain variants such as BoNT/A, BoNT/B, BoNT/C₁, BoNT/D, BoNT/E, BoNT/F or BoNT/G light chain variants or tetanus toxin (TeNT) light chain variants.

A variety of populations can be assayed according to a method of the invention including, without limitation, random populations. In one embodiment of the invention, a population of clostridial toxin light chain variants or functional fragments thereof is produced by expressing a population of nucleic acid molecules. Genetic modification of one or more nucleic acid molecules encoding a clostridial toxin light chain or segment thereof can be useful in producing such a population of nucleic acid molecules. Genetic modifications useful in the invention include, but are not limited to, random mutagenesis, which can be used to produce, for example, a population having at least 10² different members each containing a clostridial toxin light chain variant or functional fragment thereof, or a population having at least 10³ different members each containing a clostridial toxin light chain variant or functional fragment thereof.

In one embodiment, random mutagenesis of one or more nucleic acid molecules is performed to yield an average of 1 to 3 amino acid substitutions per clostridial toxin light chain variant or functional fragment thereof. As non-limiting examples, random mutagenesis can be performed using error-prone polymerase chain reaction amplification; DNA shuffling between two or more nucleic acid molecules encoding clostridial toxin light chains or segments thereof; or saturation mutagenesis of one or more codons of one or more nucleic acid molecules encoding clostridial toxin light chains or segments thereof.

A variety of means can be useful for assaying a population for protease activity towards a selected clostridial toxin-resistant target protein in a method of the invention. In one embodiment, the invention is practiced by assaying a population of phage, each phage expressing a clostridial toxin light chain variant or functional fragment thereof. In another embodiment, the invention is practiced by assaying a population of microorganisms which each express a clostridial toxin light chain variant or functional fragment thereof. In a further embodiment, the invention is practiced by assaying a population of microorganisms which each express on the cell surface a clostridial toxin light chain variant or functional fragment thereof. Microorganisms useful in the invention encompass, without limitation, bacteria such as Escherichia coli. In yet another embodiment, the invention is practiced by selecting from the population one or more viable members which each contain an evolved clostridial toxin light chain or functional fragment thereof.

The invention also can be practiced by assaying a population of purified or partially purified polypeptides, or functional fragments thereof, for protease activity towards a selected clostridial toxin-resistant target protein. Such a population can be, for example, a population of purified clostridial toxin light chain variants or functional fragments thereof. Such a population also can be a population of purified toxins, which contain a clostridial toxin heavy chain and a clostridial toxin light chain variant. In one embodiment, the invention is practiced by assaying a population of purified dichain toxins.

A variety of techniques can be useful for assaying for protease activity towards a selected clostridial toxin-resistant target protein. Such techniques include, yet are not limited to, immunoassays such as enzyme-linked immunosorbent assays, fluorescence resonance energy transfer assays, and fluorescence activated cell sorting assays. In one embodiment, the steps of the invention are repeated one or more times. In other embodiments, the steps of the invention are repeated two or more times or three or more times.

In the methods of the invention, one or more clostridial toxin light chains can be selected as a “starting point” for evolution based on the desired altered protease specificity or other enzymatic properties desired in the evolved light chain. Such selected clostridial toxin light chains include wild type clostridial toxin light chains as isolated from any serotype of Clostridia as well as mutant clostridial toxin light chains that differ from a wild type clostridial toxin light chain by one or more amino acids and have, for example, a useful characteristic. The one or more clostridial toxin light chains selected as a starting point for preparation of a population of variants can be any clostridial toxin light chain including, but not limited to, wild type and non-naturally occurring forms of BoNT/A, BoNT/B, BoNT/C₁, BoNT/D, BoNT/E, BoNT/F, BoNT/G and TeNT. Nucleic acid and corresponding amino acid sequences for wild type clostridial toxins are well known in the art and available, for example, under Genbank accession X52066 (BoNT/A); M81186 (BoNT/B); X66433 (BoNT/C1); X54254 (BoNT/D); X62088 (BoNT/E); M92906 (BoNT/F); X74162 (BoNT/G); and X04436 (TeNT). See, also, Binz et al., J. Biol. Chem. 265:9153-9158 (1990). The skilled person understands that, due to the degeneracy of the genetic code, a variety of different nucleic acid sequences encoding the same or similar amino acid sequences can be useful as “parent sequences” for the preparation of a population of clostridial toxin light chain variants or functional fragments thereof.

As an example, BoNT/A and BoNT/E cleave human SNAP-25; where the clostridial toxin-resistant target protein is the related protein, human SNAP-23, the population of clostridial toxin light chain variants can be, for example, a population of BoNT/A variants or a population of BoNT/E variants or a mixture thereof. It is understood that, if desired, two or more nucleic acid molecules encoding selected clostridial toxin light chains or segments thereof can serve as the one or more “parent sequences” that are subject to genetic modification such as random mutagenesis to generate a population of clostridial toxin light chain variants or functional fragments thereof. It further is understood that, where multiple iterations of a method of the invention are performed, the population of clostridial toxin light chain variants or functional fragments used in a second or subsequent iteration can be generated based on the sequence of an evolved clostridial toxin light chain isolated in a preceding iteration, for example, by random mutagenesis of the sequence encoding an evolved clostridial toxin light chain isolated in a preceding iteration.

The methods of the invention involve assaying a population having members that each contain a clostridial toxin light chain variant or functional fragment thereof. As used herein, the term “variant” means a clostridial toxin light chain having a non-naturally occurring amino acid sequence that differs at one or more amino acid positions from the sequence of a naturally occurring clostridial toxin light chain. Variants differ from naturally occurring light chains by some detectable structural property such as a difference in at least one amino acid residue or a difference introduced by the modification of an amino acid such as the addition of a chemical functional group. A clostridial toxin light chain variant can have an amino acid sequence that is more closely related to the sequence of one particular naturally occurring clostridial toxin light chain than to other naturally occurring clostridial toxin light chains; where this is the case, it may be designated, for example, a “BoNT/A variant.”

The methods of the invention also can be practiced with a functional fragment of a clostridial toxin light chain variant. As used herein, the term “functional fragment” means a portion of a full-length clostridial toxin light chain variant, where the portion corresponds to that part of a wild type light chain that retains proteolytic activity for its cognate, toxin-sensitive target protein. Thus, a “functional fragment” may or may not have proteolytic activity; however, the corresponding portion of a wild type light chain has proteolytic activity for its cognate target protein. The term “functional fragment” is contrasted herein with the term “segment,” as described further below.

Functional fragments of wild type clostridial toxin light chains are known in the art. As examples, fragments having residues 9-447, 1-425, 1-420, 1-406 and 9-415 of the wild type BoNT/A light chain have been shown to retain activity as have fragments having residues 9-447, 1-398, 1-392 and 1-389 of the wild type TeNT light chain (Kurazono et al., J. Biol. Chem. 267:14721-14729 (1992); and Kadkhodayan et al., Prot. Exp. Purif. 19:125-130 (2000)). Thus, a functional fragment useful in the invention can be, for example, a fragment of about 390 to 430 residues or a fragment of about 400 to 420 residues. In particular embodiments, a functional fragment has at most 150, 200, 250, 300, 350 or 400 residues, or at least 150, 200, 250, 300 or 350 residues. In further embodiments, a functional fragment corresponds to residues 9-447, 9-425, 9-420 or 9-406 of a clostridial toxin light chain. In still further embodiments, a functional fragment has residues 9-447, 9-425, 9-420 or 9-406 of a BoNT/A or BoNT/E light chain.

As used herein, the term “population” means a group of two or more different members that each include a clostridial toxin light chain variant or functional fragment thereof. It is understood that a member can be physically associated with a single clostridial toxin light chain variant or functional fragment or an encoding nucleic acid molecule, or can be physically associated with two or more distinct molecular species of clostridial toxin light chain variant or functional fragment or nucleic acid molecules encoding two or more distinct molecular species of light chain variant or functional fragment. The members of a population, where present, can serve, for example, to physically link a clostridial toxin light chain variant or functional fragment with the corresponding encoding nucleic acid molecule. As non-limiting examples, a member can be: the clostridial toxin light chain variant or functional fragment itself; a phage displaying a clostridial toxin light chain variant or functional fragment thereof; a virus expressing a clostridial toxin light chain variant or functional fragment thereof; a cell or organism such as, for example, an E. coli, yeast, baculovirus or other insect cell, or mammalian cell expressing intracellularly or on the cell surface a clostridial toxin light chain variant or functional fragment thereof; a nucleic acid molecule linked to a clostridial toxin light chain variant or functional fragment thereof; a polypeptide such as a component of a three-hybrid system; a bead; a liposome; a lipid vesicle, agarose gel or other microdroplet; a gel-like matrix; or another particle, organism or microdevice stably associated with one or more distinct clostridial toxin light chain variants or functional fragments thereof. In one embodiment, a method of the invention is practiced with a population containing members which are each physically associated with a single molecular species of clostridial toxin light chain variant or functional fragment thereof.

As set forth above, the members of a population each include a clostridial toxin light chain variant or functional fragment thereof. In one embodiment, the members of a population each include a clostridial toxin light chain variant or functional fragment thereof in the absence of a clostridial toxin heavy chain. In another embodiment, the members of a population each include a clostridial toxin light chain variant or functional fragment thereof and further include a clostridial toxin heavy chain. Such a heavy chain can be, for example, the heavy chain most closely related to the clostridial toxin light chain variant included in the member and further can be a wild type or modified heavy chain.

A variety of populations are useful in the methods of the invention; such populations typically are of sufficient size and diversity so as to contain at least one member that includes a clostridial toxin light chain variant, or functional fragment thereof, which has protease activity towards the selected clostridial toxin-resistant target protein. Populations useful in the invention can be, for example, as small as two members having at least one clostridial toxin light chain variant or functional fragment thereof, and as large as, for example, 10¹⁵ members having at least one clostridial toxin light chain variant or functional fragment thereof. In particular embodiments, the methods of the invention are practiced with a population having between five and 20 members, each having at least one clostridial toxin light chain variant or functional fragment thereof; a population having at most 100 members, each including at least one clostridial toxin light chain variant or functional fragment thereof; or a population having at most 1000 members, each including at least one clostridial toxin light chain variant or functional fragment thereof. In other embodiments, the methods of the invention are practiced with a population having at most 10⁴, 10⁵ or 10⁶ members, each including at least one clostridial toxin light chain variant or functional fragment thereof. In further embodiments, the methods of the invention are practiced with a population having more than 10⁴, 10⁵ or 10⁶ members, each member including at least one clostridial toxin light chain variant or functional fragment thereof. In yet another embodiment, the methods of the invention are practiced with a population having between 10⁶ and 10⁸ members, each having at least one clostridial toxin light chain variant or functional fragment thereof.

It is understood that the same variants or functional fragments can be represented by members of the population two or more times; “complexity” is a term that denotes the number of different molecular species included in a population. It further is understood that a given population can include some members containing variants or functional fragments having, for example, a single amino acid substitution relative to a naturally occurring clostridial toxin together with other members containing variants or functional fragments having multiple amino acid substitutions and, if desired, can favor the over-representation of some molecular species or classes of species relative to other species. In particular embodiments, a population useful in the invention has members including at most 100 different clostridial toxin light chain variants or functional fragments, or at most 1000, 10⁴, 10⁵, 10⁶, 10⁷, 10⁸, 10⁹, 10¹⁰, 10¹¹, 10¹², 10¹³, 10¹⁴ or 10¹⁵ different clostridial toxin light chain variants or functional fragments. In further embodiments, a population useful in the invention has members including at least 100 different clostridial toxin light chain variants or functional fragments, or at least 1000, 10⁴, 10⁵, 10⁶, 10⁷, 10⁸, 10⁹, 10¹⁰, 10¹¹, 10¹², 10¹³, 10¹⁴ or 10¹⁵ different clostridial toxin light chain variants or functional fragments.

In a population useful in the invention, the clostridial toxin light chain variants or functional fragments thereof can have, for example, substitutions at a single amino acid position relative to a naturally occurring clostridial toxin light chain, where the substitutions are changes to all amino acids that do not occur naturally at this position in the selected clostridial toxin light chain. In this example, the population would have nineteen different members, each different member having a clostridial toxin light chain variant with a different amino acid substitution at a single amino acid position. A population useful in the invention also can contain members in which the clostridial toxin light chain variants or functional fragments thereof have, for example, at least one amino acid substitution at two or more distinct amino acid positions relative to a naturally occurring clostridial toxin light chain. In this example, a minimal population would have two different members, each member including a clostridial toxin light chain variant or functional fragment having an amino acid substitution at one of two distinct positions. It is understood that such a population can be expanded with the addition of substitutions to all of the 19 non-naturally occurring amino acids at the two amino acid positions or additional amino acid positions.

In one embodiment, a population useful in the invention contains members in which the clostridial toxin light chain variants or functional fragments thereof all have at most 20 amino acid substitutions relative to the same wild type clostridial toxin light chain. In another embodiment, a population useful in the invention contains members in which the clostridial toxin light chain variants or functional fragments thereof all have at most 10 amino acid substitutions relative to the same wild type clostridial toxin light chain. In further embodiments, a population useful in the invention contains members in which the clostridial toxin light chain variants or functional fragments thereof all have at most 9, 8, 7, 6, 5, 4, 3 or 2 amino acid substitutions relative to the same wild type clostridial toxin light chain. In still further embodiments, a population useful in the invention contains members in which the clostridial toxin light chain variants or functional fragments thereof all have at least 30%, 40%, 50%, 60%, 70%, 75%, 80%, 85%, 90% or 95% amino acid identity relative to the same wild type clostridial toxin light chain.

The methods of the invention rely on assaying for protease activity towards a selected clostridial toxin-resistant target protein. Protease activity is assayed using a “selected substrate,” which has the same cleavage site and generally the same recognition sequence as the selected clostridial toxin-resistant target protein; such a recognition sequence is a scissile bond together with adjacent or non-adjacent recognition elements sufficient for detectable proteolysis at the scissile bond under conditions suitable for clostridial toxin protease activity. Such a recognition sequence can be the region corresponding to the minimum binding domain of a clostridial toxin-sensitive target protein. Examples of selected substrates include the selected clostridial toxin-resistant target protein itself, portions thereof, or synthetic peptides or peptidomimetics that serve to assay for protease activity towards the selected clostridial toxin-resistant target protein, as described further below. The extent of proteolysis of a selected substrate correlates with proteolysis of the selected clostridial toxin-resistant target protein and, thus, the selected substrate serves as a surrogate for the selected target protein. It is understood that the methods of the invention can be performed with crude, partially purified or purified substrates; purified substrates include, without limitation, recombinant, chemically synthesized and biochemically purified protein and peptides.

Selected substrates useful in the invention include, without limitation, peptidomimetics. As used herein, the term “peptidomimetic” is used broadly to mean a peptide-like molecule that is cleaved by the same clostridial toxin light chain as the peptide substrate upon which it is structurally based. Such peptidomimetics include chemically modified peptides, peptide-like molecules containing non-naturally occurring amino acids, and peptoids, which are peptide-like molecules resulting from oligomeric assembly of N-substituted glycines. Peptidomimetics useful in the invention include, without limitation, peptide-like molecules which contain a constrained amino acid, a non-peptide component that mimics peptide secondary structure, or an amide bond isostere. See, for example, Goodman and Ro, Peptidomimetics for Drug Design, in “Burger's Medicinal Chemistry and Drug Discovery” Vol. 1 (ed. M. E. Wolff; John Wiley & Sons 1995), pages 803-861.

The methods of the invention can be practiced, if desired, by genetic modification of one or more nucleic acid molecules encoding clostridial toxin light chains or segments thereof. As used herein in reference to a clostridial toxin light chain, the term “segment” means a portion of a full-length clostridial toxin light chain. In contrast to a functional fragment, the portion of a wild type light chain that corresponds to a “segment” may or may not have proteolytic activity. A segment can be, for example, a piece of a functional fragment. As an example, a nucleic acid molecule encoding a segment of a clostridial toxin light chain can be flanked by convenient restriction enzyme sites and, after being genetically modified, the nucleic acid molecule encoding the “modified segment” can be substituted for the corresponding wild type sequence within a nucleic acid molecule encoding a full-length clostridial toxin light chain, or within a nucleic acid molecule encoding a functional fragment, to produce a clostridial toxin light chain variant or functional fragment thereof.

Genetic modification, which is any process whereby one or more changes occur in the structure of a nucleic acid molecule, can arise spontaneously or can be induced. Genetic modification can involve alteration of the nucleic acid sequence of a single gene or portion thereof, alteration of blocks of genes, or whole chromosomes. Changes in single genes can be the consequence of point mutations involving the removal, addition or substitution of a single nucleotide base within a nucleic acid sequence, or can be the consequence of changes involving the insertion, deletion or substitution of small or large numbers of nucleotides.

In one embodiment, the methods of the invention are practiced by random mutagenesis of one or more nucleic acid molecules encoding clostridial toxin light chains or segments thereof. Where the randomly mutagenized nucleic acid molecules do not encode a full-length light chain or functional fragment thereof, the nucleic acid molecules generally are subcloned into the appropriate larger nucleic acid molecule to produce a population of nucleic acid molecules encoding a population of clostridial toxin light chain variants or a population of functional fragments.

As used herein, the term “random mutagenesis” means a process whereby a change in the structure of a nucleic acid molecule is produced, where the change is not a change to a single predetermined residue at a predetermined position. Random mutagenesis can be performed by a variety of well known methods including, but not limited to, enzymatic methods, chemical methods and physical methods, and further including, without limitation, saturation mutagenesis at one or more amino acid positions. As non-limiting examples, random mutagenesis can be performed using error-prone PCR or another method that relies on errors in the fidelity of DNA replication; degenerate oligonucleotide site-specific mutagenesis; insertional mutagenesis, for example, using transposable genetic elements (transposons); recombination-based methods such as DNA shuffling; mutagenic organisms such as mutant E. coli strains; and physical or chemical methods based on mutagens including, without limitation, ionizing radiation, ultraviolet light, and chemical mutagens such as alkylating agents and polycyclic aromatic hydrocarbons.

Random mutagenesis can be performed, if desired, throughout a nucleic acid molecule encoding a clostridial toxin light chain or functional fragment thereof in order to identify amino acid residues critical for protease function. Segments containing these critical amino acid residues are target sequences for introducing random mutations to produce an evolved clostridial toxin light chain having altered protease specificity. Methods for identifying critical amino acid residues by introducing a small number of random mutations throughout a gene segment are well known to those skilled in the art and include, for example, copying by mutagenic polymerases, exposure of templates to DNA damaging agents, and replacement of regions of the nucleic acid template with oligonucleotides containing sparsely populated random inserts.

As indicated above, error-prone polymerase chain reaction amplification can be useful for performing random mutagenesis in the methods of the invention. Error-prone PCR is well known in the art as described, for example, in Cadwell and Joyce, PCR Methods and Applications 2:28 (1992); the rate of mutagenesis can be enhanced, if desired, by performing PCR in multiple tubes with different template dilutions or with varying magnesium concentrations.

Saturation mutagenesis is a form of random mutagenesis in which all 19 amino acid substitutions are produced at one or more amino acid positions within a clostridial toxin light chain or segment thereof. Saturation mutagenesis on numerous residues, sometimes known as in vitro scanning saturation mutagenesis, can be performed by routine methods (Burks et al., Proc. Natl. Acad. Sci., USA 94:412-417 (1997); and Miyazaki and Arnold, J. Mol. Evol. 49:716-720 (1999).

Random mutagenesis also can be performed using degenerate oligonucleotides. A double-stranded oligodeoxyribonucleotide can be produced by hybridization of two partially complementary oligonucleotides, one or both of which contain random sequences at specified positions. The partially double-stranded oligonucleotide can be filled in by DNA polymerase, digested at convenient restriction sites and substituted for the corresponding region of a nucleic acid molecule encoding a full-length clostridial toxin light chain or a functional fragment thereof. After ligation, the reconstructed expression vectors constitute a population of nucleic acid molecules encoding a population of clostridial toxin light chain variants or functional fragments thereof.

A variety of additional genetic means also can be used to create a population of members containing clostridial toxin light chain variants or functional fragments thereof. Such genetic means can be used, for example, to recombine mutations such as beneficial mutations found in early rounds of assaying for protease activity towards a selected clostridial toxin-resistant target protein. Such methods include, yet are not limited to, DNA shuffling; staggered extension process (StEP) in vitro recombination; and subdomain shuffling as described, for example, in Stemmer, Nature 370: 389-391 (1994); Crameri et al., Nature 391:288-291 (1998); Zhao et al., Nature Biotech. 16:258-261 (1998); Ostermeier et al., Nature Biotech. 17:1205-1209 (1999); Hopfner et al., Proc. Natl. Acad. Sci., USA 95:9813-9818 (1998); and Lutz and Benkovic, Curr. Opin. Biotech. 11:331-337 (2000). Additional hybrid populations also can be useful in the methods of the invention. As an example, incremental truncation for the creation of hybrid enzymes (ITCHY) using 5′ fragments encoding a segment of a first clostridial toxin light chain, such as BoNT/A, and 3′ fragments encoding a segment of a second clostridial toxin light chain such as BoNT/E, also can be useful for generating a population to be assayed for protease activity in a method of the invention (Ostermeier et al. Supra, 1999). The skilled person understands that these and a variety of additional procedures for genetic modification can be useful for generating a population in the methods of the invention.

Radiation mutagenesis also can be useful for random mutagenesis in a method of the invention. Radiation mutagenesis is the use of particles or photons having sufficient energy or that can produce sufficient energy through nuclear interactions to produce ionization, which is the gain or loss of electrons. In one embodiment, the radiation mutagenesis is performed using X-radiation. Where a cell is mutagenized, the amount of ionizing radiation to be used depends, in part, on the nature of the cell type, and typically is less than the dose of ionizing radiation that causes cell damage or death. The amount of ionizing radiation administered to a cell to perform random mutagenesis according to a method of the invention can be, for example, from 2 to 30 Gray (Gy), or from 5 to 15 Gy, administered at a rate of from 0.5 to 2 Gy/minute. In other embodiments, the dose of ionizing radiation administered to a cell to perform random mutagenesis is from 10 to 100 Gy, from 15 to 75 Gy, or from 20 to 50 Gy.

Chemical mutagenesis is another form of random mutagenesis useful in the invention. Chemical mutagenesis can be performed, for example, with a chemical carcinogen. As non-limiting examples, Benzo[a]pyrene, N-acetoxy-2-acetyl aminofluorene, and aflotoxin B1 can produce GC to TA transversions in bacteria and mammalian cells; benzo[a]pyrene also can produce base substitutions such as AT to TA substitutions; N-nitro compounds can produce GC to AT transitions; and N-nitrosoureas can produce TA to CG transitions through alkylation of the O4 position of thymine. In addition, N-nitroso-N-methyl urea is useful for chemical mutagenesis of eukaryotic cells. The skilled person understands that these and other chemical mutagens can be useful for producing a population of nucleic acid molecules encoding a population of clostridial toxin light chain variants or functional fragments to be assayed for protease activity in a method of the invention.

A method of the invention involves, in part, assaying a population of members, each including a clostridial toxin light chain variant or functional fragment thereof, for protease activity towards a selected clostridial toxin-resistant target protein. A variety of assays are useful in the methods of the invention including, without limitation, solid phase and solution-based assays.

Phenotypic selections for obligate activity can be useful in the methods of the invention. In one embodiment, a method of the invention is practiced by selecting from a population one or more viable members, where each viable member includes an evolved clostridial toxin light chain or functional fragment thereof. As used herein, the term “selection” is synonymous with “obligate activity selection” and means a separation process based on preferential survival of particular organisms, in this case preferential survival of organisms containing a clostridial toxin light chain variant or functional fragment thereof with protease activity towards the selected clostridial toxin-resistant target protein. Such selections generally are based on complementation of auxotrophy or resistance to a cytotoxic agent such as an antibiotic and include selectable phenotypes which are directly or indirectly linked to proteolysis of the selected clostridial toxin-resistant target protein.

Assays for protease activity towards a selected clostridial toxin-resistant target protein can be performed in a variety of formats including growth of microorganisms such as bacteria or yeast on a solid substrate, for example, agar. Assays for protease activity further include those based on a fluorescent cleavage product, immunologically detectable cleavage product, or otherwise detectable cleavage product. In a population of cells such as unicellular microorganisms, each cell is physically associated with one or more clostridial toxin light chain variants or functional fragments thereof. Where the variants or functional fragments are expressed intracellularly, a protease assay can be performed with viable or intact cells using a fluorescence resonance energy transfer (FRET) assay as described, for example, in U.S. Ser. No. 10/261,161, or can be performed following lysis of the cell. A population of microorganisms also can be assayed by “replica plating,” or transferring a portion of each colony to, for example, a filter membrane; the transferred portion can be lysed and assayed while the remaining portion serves to isolate the evolved clostridial toxin light chain or functional fragment thereof (Matsumara et al., Nature Biotech. 17:696-701 (1999)). A population of members, such as phage or cells, for example, bacterial, microbial, yeast, insect or mammalian cells, also can be physically associated, whether directly or indirectly, with a selected substrate in addition to the one or more clostridial toxin light chain variants or functional fragments thereof. Examples of such “proximity coupling” methodologies are described further hereinbelow.

In a population useful in the invention, a variety of cells can serve as members that can express or otherwise be physically associated with a clostridial toxin light chain variant or functional fragment thereof. Such cells include prokaryotic and eukaryotic cells and encompass, without limitation, bacterial cells, yeast cells, insect cells such as baculovirus cells, and mammalian cells, including murine, rat, primate and human cells. Methods for introducing a nucleic acid molecule into a host cell for expression of a clostridial toxin light chain variant or functional fragment are well known in the art and depend, in part, on the type of cell; such methods include, without limitation, electroporation, microinjection, calcium phosphate, DEAE-dextran, lipofection and viral-based methods (see, for example, Ausubel, supra, 2000).

Microorganisms that can express or otherwise be physically associated with a clostridial toxin light chain variant or functional fragment include, yet are not limited to, bacteria such as Gram-negative bacteria, for example, E. coli, and further encompass, without limitation, Salmonella, Klebsiella, Erwinia, Pseudomonas aeruginosa, Haemophilus influenza, Rickettsia rickettsii, and Neisseria gonorrhea. In one embodiment, a method of the invention is practiced by assaying a population of microorganisms, each microorganism expressing on its surface a clostridial toxin light chain variant or functional fragment thereof. Flow cytometry coupled with cell-surface display is a quantitative method amenable to high throughput analysis of such microorganisms, with up to 10⁹ cells/hour readily assayed. In a method of the invention, a population of microorganisms can be assayed for protease activity using, for example, cell sorting or fluorescence activated cell sorting (FACS). In one embodiment, the population of microorganisms is a population of Gram-negative bacteria, which have a negatively charged surface. Cell surface displayed polypeptide libraries useful for cell sorting can be prepared by routine methods. See, for example, Daugherty et al., “Flow cytometric screening of cellular combinatorial libraries,” J. Immunol. Methods 243: 211-227 (2000); Holler et al., Proc. Natl. Acad. Sci., USA 97: 5387-5392 (2000); U.S. Pat. No. 5,348,867; and Olsen et al., Nature Biotech. 18:1071-1074 (2000).

For expression on the surface of a Gram-negative bacteria, a clostridial toxin light chain variant, or functional fragment thereof, can be fused to an amino acid sequence that includes signals sufficient for localization to the outer membrane and for translocation across the outer membrane, with the sequences responsible for localization and translocation derived from the same or different proteins and from the same or different species. As a non-limiting example, a clostridial toxin light chain variant or functional fragment thereof can be fused to a portion of a major lipoprotein and OmpA by well known methods; surface expression vehicles such as the LPP-OmpA system are described, for example, in Francisco et al., Proc. Natl. Acad. Sci., USA 89:2713-2717 (1992); and Francisco et al., Biotech. 11:491-496 (1993). Translocation can be achieved, for example, with an E. coli OmpA, LamB, PhoE, OmpC, OmpF, OmpT or FepA protein or an equivalent of one of these proteins such as a Salmonella equivalent. Outer membrane targeting sequences include, without limitation, those derived from E. coli Lpp, TraT, OsmB, N1pB, OprI, or BlaZ; Pseudomonas aeruginosa Lpp1; Haemophilus influenze PA1; Rickettsia rickettsii 17 kDa 1pp or a Neisseria gonorrhea H.8 protein, or a species homolog or other equivalent of one of these proteins. Such outer membrane targeting sequence are well known in the art as described, for example, in WO 98/49286.

Yeast are another microorganism useful for surface expression of a clostridial toxin light chain variant or functional fragment thereof. Yeast surface display expression systems, including surface display of agglutinin fusion proteins, are well known in the art. See, for example, Schreuder et al., Trends Biotech. 14:115-120 (1996); Schreuder et al., Vaccine 14:383-388 (1996); and Boder and Wittrup, Nature Biotech. 15:553-557 (1997).

In one embodiment, the invention is practiced with a population of cells that each include a selected substrate physically associated with the cell surface in addition to expressing a clostridial toxin light chain variant or functional fragment thereof. In another embodiment, the invention is practiced with a population of Gram-negative bacteria which each include a selected substrate that is physically associated with the bacterial cell surface via the substrate's polycationic tail. In a further embodiment, the invention is practiced using fluorescence activated cell sorting to assay a population of cells expressing on the cell surface a fluorescent substrate and a clostridial toxin light chain variant or functional fragment thereof, where the fluorescent substrate exhibits fluorescence resonance energy transfer (FRET). See, for example, Olsen et al., Curr. Opin. Biotech. 11:331-337 (2000). Such a population can be a population of microorganisms such as bacteria.

Thus, in particular embodiments, protease activity towards a selected clostridial toxin-resistant target protein is assayed using a population of cells expressing on the cell surface a FRET substrate and a clostridial toxin light chain variant or functional fragment thereof. Such a FRET substrate includes a donor fluorophore; an acceptor having an absorbance spectrum overlapping the emission spectrum of the donor fluorophore, and a clostridial toxin-resistant recognition sequence including a cleavage site. The cleavage site intervenes between the donor fluorophore and the acceptor such that, under the appropriate conditions, resonance energy transfer is exhibited between the donor fluorophore and the acceptor. In one embodiment, the FRET substrate includes a non-fluorescent acceptor, sometimes known as a “quencher.” In this case, proteolytic cleavage of the selected substrate at the intervening cleavage site gives rise to a fluorescent product such that the amount of fluorescence correlates with the extent of protease activity at the cleavage site. In one embodiment, the fluorescent product is retained on the surface of the cell so that fluorescence activated cell sorting can be used to isolate the cell expressing the evolved clostridial toxin light chain exhibiting protease activity towards the selected clostridial toxin-resistant target protein. In the case of Gram-negative bacteria, the fluorescent product can be conveniently retained on the surface of the microorganism by a polycationic tail.

A population useful in the invention also can be a population of viruses such as phage and further can be, without limitation, a population of filamentous phage. In one embodiment, the invention provides a method of isolating an evolved clostridial toxin light chain having altered protease specificity using a population of phage each expressing on the surface a clostridial toxin light chain variant or functional fragment thereof. Phage display is well known in the art for expression of libraries useful in iterative mutagenesis and screening or selection strategies (Olsen et al., supra, 2000). A variety of means can be used to express a clostridial toxin light chain variant or functional fragment thereof on the surface of a filamentous phage. In one embodiment, the clostridial toxin light chain variant or functional fragment thereof is expressed as a fusion with the phage pIII protein. In other embodiments, the clostridial toxin light chain variant or functional fragment thereof is expressed as a fusion with the phage pVII or pIX proteins. In yet a further embodiment, the clostridial toxin light chain variant or functional fragment thereof is expressed as a fusion with the phage pVIII protein. One skilled in the art understands that pIII, PVII and pIX fusions generally are useful for low copy number display, for example, 1-5 copies of fusion protein per phage particle; pVIII fusions generally are useful for high copy number display, with greater than 100 copies of the fusion protein expressed per phage particle. Methodologies for phage-display are well known in the art as described, for example, in Forrer et al., Curr. Opin. Struct. Biol. 9:514-520 (1999); and Sidhu et al., J. Mol. Biol. 296:487-495 (2000).

A method of the invention can be practiced, if desired, using a population of phage each expressing a clostridial toxin light chain variant or functional fragment thereof, and further expressing a selected substrate. As described above, such a selected substrate has the same cleavage site and generally the same recognition sequence as the selected clostridial toxin-resistant target protein, and can be the target protein itself, a segment thereof, or a synthetic peptide or peptidomimetic that serves to assay for protease activity towards the selected clostridial toxin-resistant target protein. Proteolysis of the selected substrate displayed on a phage can be detected, for example, by selective release or retention of phage particles following substrate cleavage. In one embodiment, a phage population displaying both a clostridial toxin light chain variant or functional fragment thereof and a selected substrate is soluble prior to proteolysis of the selected substrate and is immobilized on a solid support subsequent to proteolysis. In another embodiment, a phage population displaying both a clostridial toxin light chain variant or functional fragment thereof and a selected substrate is immobilized on a solid support prior to proteolysis of the selected substrate and is released from the solid support subsequent to proteolysis.

As a non-limiting example, a phage population useful in the invention can express a pIII-clostridial toxin light chain variant, or functional fragment, fusion polypeptide and can further express a selected substrate through covalent alkylation of pVIII. Such a population is soluble prior to proteolysis; after proteolysis, phage expressing cleavage product can be selectively bound on a solid support, for example, using immunoaffinity chromatography involving specific recognition of the cleavage product. Methods of physically associating substrate with a phage, for example, through non-specific alkylation of the major outer coat protein gene VIII are well known in the art as described, for example, in Jestin et al., Angew Chem. Int. Ed. 38:1124-1127 (1999).

As a further non-limiting example, a phage population useful in the invention can express a pIII-clostridial toxin light chain variant, or functional fragment, fusion polypeptide as well as a pIII-substrate fusion. Such a chimeric phage population can be retained, for example, on a streptavidin-coated solid support through a biotin tag fused to the substrate. Proteolysis can be detected through release of phage from the streptavidin support as described in Pedersen et al., Proc. Natl. Acad. Sci., USA 95:10523-10528 (1998).

As an additional non-limiting example, a phage population useful in the invention can express a pIII-calmodulin-clostridial toxin light chain variant, or functional fragment, fusion polypeptide that includes flexible linkers between the pIII protein and variant or functional fragment; a selected substrate can be associated with the phage, for example, through a calmodulin-binding peptide. While the phage are initially soluble, proteolysis of the calmodulin-bound phage-substrate conjugate can be detected by phage retention on a solid support using an antibody or other binding agent that specifically binds cleavage product. See, for example, Demartis et al., J. Mol. Biol. 286:617-633 (1999). It is understood that similar affinity couples can be equivalently substituted for calmodulin/calmodulin-binding peptide in this or a related assay useful in the methods of the invention.

A population useful in the invention further can be a population of cells or microorganisms which each contain a nucleic acid molecule encoding a clostridial toxin light chain variant, or functional fragment thereof, and which secrete the variant or functional fragment. Populations useful for secretion of clostridial toxin light chain variants include, without limitation, E. coli or other bacterial populations; populations of yeast such as S. cerevisiae or P. pastoris; baculovirus or other insect cell populations; and populations of mammalian cells. Systems for expression and secretion of heterologous proteins are well known in the art and commercially available. As non-limiting examples, the pBAD-gIII (Invitrogen) and pET (Novagen) E. coli expression systems can be useful in the invention. Where clostridial toxin light chain variants or functional fragments are expressed in E. coli (periplasmic space), the cells can be concentrated by centrifugation prior to releasing the clostridial toxin light chain variant or functional fragment using osmotic shock.

A population useful in the invention also can be a population of clostridial toxin light chain variants or functional fragments displayed on nucleic acid molecules such as mRNA. The nucleic acid molecules can encode the covalently linked clostridial toxin light chain variant or functional fragment or can serve as a non-coding genetic tag to uniquely identify the covalently linked light chain variant or functional fragment thereof. Such nucleic acid molecules can include common primer binding sites for PCR amplification of the linked nucleic acid molecule. Such mRNA and other nucleic acid based display systems are well known in the art and are commercially available, for example, the PROfusion™ technology from Phylos, Inc. (Lexington, Mass.). See, also, Roberts, Curr. Opin. Chem. Biol. 3: 268-273 (1999); Roberts and Szostak, Proc. Natio. Acad. Sci., USA 94:1297-12302 (1997); and Kreider, Med. Res. Rev. 20:212-215 (2000).

A variety of additional means are available to the skilled person for generating a population of members that each include a clostridial toxin light chain variant or functional fragment thereof. Members that can be physically associated with a clostridial toxin light chain variant or functional fragment thereof for use in the invention encompass nucleic acid molecules, cells, organisms and other biological and non-biological members, including, without limitation, lipid vesicles (Tawfik and Griffiths, Nature Biotech. 16:652-656 (1998)); agarose gel microdroplets; and components of a three hybrid system as described, for example, in Firestine et al., Nature Biotech. 18:544-547 (2000). As a non-limiting example, reverse micelles can be produced by dispersing an in vitro transcription-translation mixture in, for example, a water-in-oil emulsion; the reverse micelles can be prepared under conditions chosen such that an average of one light chain variant or functional fragment-encoding nucleic acid molecule is incorporated per micelle.

An agarose gel microdroplet (AGM) also can be a member useful in a method of the invention. Agarose gel microdroplets are micron sized particles which can be sized to include one initial cell or colony forming unit. As an example, a conventional cell suspension can be used to generate a large number (10⁶/ml) of agarose gel microdroplets by adding the cells to molten agarose and subsequent dispersion into mineral oil. After the agarose gel microdroplet suspension is transiently cooled to a gelatin state, poisson statistics allow the determination of the size of those microdroplets having a high probability of containing zero or one initial cell or colony forming unit. The microdroplets are transferred out of the mineral oil into a suitable growth medium and incubated to allow formation of microcolonies, which can be stained with a dye for one or more generic indicators of biomass such as propidium iodide or FITC and then assayed using, for example, flow cytometry. Such a method is applicable to any cell type amenable to being cultured in a gel-like matrix, including mammalian, fungal and bacterial cells. See, for example, WO 98/49286 and Weaver et al., Biotechnology 9:873-877 (1991). The skilled person understands that these and a variety of other types of populations can be useful in the methods of the invention.

Any of a variety of techniques can be useful in a method of the invention for assaying for protease activity towards a selected clostridial toxin-resistant target protein. Such techniques include, without limitation, immunoassays such as enzyme-linked immunosorbent assays, fluorescence resonance energy transfer assays, and fluorescence activated cell sorting assays. Additional assays further include direct quantitation of substrate cleavage products. As an example, BoNT/A enzyme activity has been analyzed by HPLC separation and quantitation of peptide substrate hydrolysis products (Schmidt and Bostian, J. Prot. Chem. 14:703-708 (1995)).

One skilled in the art understands that multiple iterations of generating variants or functional fragments, assaying for protease activity towards the selected clostridial toxin-resistant target protein, and isolating one or more members including an evolved clostridial toxin light chain can be useful in the invention. In particular embodiments, the invention is practiced using at least two, at least three, at least four, at least five, at least ten, at least fifteen, at least 20, or at least 25 iterations to produce an evolved clostridial toxin light chain or functional fragment thereof. In further embodiments, the invention is practiced using two, three, four, five, six, seven, eight, nine or ten iterations to produce an evolved clostridial toxin light chain or functional fragment thereof. One skilled in the art understands that the isolated members, such as cells, phage or microorganisms, exhibiting protease activity towards the selected target protein can optionally be regrown between iterations.

A method of the invention can further optionally include assaying the population of members for binding activity against a selected clostridial toxin-resistant target protein or selected substrate. In one embodiment, a method of the invention is practiced by (a) generating a population, each member of which contains a clostridial toxin light chain variant or functional fragment thereof; (b) assaying the population of members for binding activity against a selected clostridial toxin-resistant target protein; (c) isolating from the population one or more members with binding activity to form an enriched population (d) assaying the enriched population for protease activity towards said selected clostridial toxin-resistant target protein, where increased protease activity is indicative of an evolved clostridial toxin light chain; and (e) isolating from the enriched population one or more members that contain an evolved clostridial toxin light chain or functional fragment thereof.

In another embodiment, a method of the invention is practiced by (a) generating a population, each member of which contains a clostridial toxin light chain variant or functional fragment thereof; (b) assaying the population of members for binding activity against a selected clostridial toxin-resistant target protein; (c) isolating from the population one or more members with binding activity to form an enriched population; generating a further population, each member of which contains a clostridial toxin light chain variant or functional fragment thereof related in structure to a member of said enriched population; (e) assaying the further population for protease activity towards said selected clostridial toxin-resistant target protein, where increased protease activity is indicative of an evolved clostridial toxin light chain; and (f) isolating from the further population one or more members that contain an evolved clostridial toxin light chain or functional fragment thereof.

In a method of the invention, the members of a population can be assayed for activity individually, in pools or en masse. Where pools of a population containing, for example, between 10 and 100 members are assayed, a pool exhibiting protease activity towards a selected clostridial toxin-resistant target protein can be subdivided, and the assay repeated in order to isolate an evolved clostridial toxin light chain or functional fragment. Some assays provide a means for “capturing” a member having protease activity and can therefore be useful in isolating an evolved clostridial toxin light chain. As an example, the members of a population can display both a substrate and a clostridial toxin light chain variant or functional fragment; upon cleavage under conditions that favor intramolecular reactions, an antibody against the appropriate cleavage product can be used to capture and isolate the evolved clostridial toxin light chain.

Immunoassays, including enzyme-linked and other immunoassays, are useful for assaying for protease activity in a method of the invention. Such assays rely on antibodies that bind a cleavage product yet do not react with intact substrate. Such methods have been generally described, for example, in U.S. Pat. No. 5,962,637. In many cases, intact substrate is immobilized on a support such as a plate; as non-limiting examples, biotin-conjugated substrates can be immobilized on a streptavidin coated support, and histidine-conjugated substrates can be immobilized on a nickel coated support.

As an example, an immunoassay useful in the invention can be performed as follows. Microtiter assay plates can be prepared by diluting peptide substrate containing a cysteine residue at one end to a final concentration of 10 μg/ml in 0.05 M sodium phosphate buffer, pH 6.5 containing 1 mM EDTA and adding the diluted peptide at 100 μl/well to a Sulphydryl Binding Plate (Costar). After incubation for one hour at room temperature, the peptide solution is removed, and the plates washed three times with phosphate buffered saline (PBS), pH 7.4. The plates are blocked by addition of PBS buffer with 0.1% Tween-20 and 5% fetal bovine serum (100 μl/well) and incubated for one hour at 37° C. with continuous shaking.

Clostridial toxin light chain variants to be assayed are diluted in an assay buffer such as 0.05 M HEPES buffer, pH 7.4, containing 10 μM ZnCl₂ and 1% fetal bovine serum with 10 mM dithiothreitol and added at 100 μl/well to the peptide-coated microtiter plates. After incubation for one hour at 37° C. with continuous shaking, the plates are washed three times with phosphate buffered saline with 0.1% Tween-20.

Antibody specific to a cleavage product, diluted in PBS buffer with 0.1% Tween-20 and 5% fetal bovine serum, is added, and the plates incubated for one hour at 37° C. with continuous shaking. Plates are washed three times with PBS containing 0.1% Tween-20. Where peroxidase-conjugated antibody is used, appropriate peroxidase substrates are added, and calorimetric results quantified.

Where unconjugated primary antibodies are used, the appropriate commercially available peroxidase-conjugated secondary antibodies are diluted in PBS with 0.1% Tween-20 and 5% FBS, added at 100 μl/well, and incubated for one hour at 37° C. with continuous shaking. After washing the plates three time with PBS containing 0.1% Tween-20, the peroxidase substrates are added and the results quantified.

Antibodies useful in such methods include monoclonal antibodies as well as affinity purified and unpurified polyclonal antiserum, which can be prepared using routine methods. As one example, for analyzing a population of variants such as BoNT/E variants for protease activity towards SNAP-23, a SNAP-23a substrate such as SEQ ID NO: 1 shown in FIG. 5, or a portion thereof, is immobilized via its amino-terminus for use as a substrate; protease activity is then analyzed with antisera prepared using the synthetic peptide EIDAQNPQIK-COOH (SEQ ID NO: 12) as an immunogen. Preparation of antibodies is well known in the art, as described, for example, in Harlow and Lane, Antibodies: A Laboratory Manual (Cold Spring Harbor Laboratory Press, 1988)). One skilled in the art understands that these and various other immunoassays, using immobilized or free protein or synthetic peptide substrates can be useful in the methods of the invention.

Assays for protease activity towards a selected clostridial toxin-resistant target protein further include fluorescence assays based on the use of a peptide substrate devoid of free amines. Peptide substrates modified to exclude free amines are exposed to a clostridial toxin light chain variant or functional fragment; the amount of proteolysis can be subsequently determined using a detectable label such as fluorescamine, which reacts with the free amine group produced by substrate cleavage, as described, for example, in U.S. Pat. No. 5,965,699.

Assays useful in the invention can be based on selected substrates in which the cleavage site for the clostridial toxin resistant target protein intervenes between an affinity tag and a detectable tag. Such selected substrates can include, for example, any of a variety of affinity tags such as 6×-HIS tags; biotin; and epitope tags such as FLAG, hemagluttinin (HA), c-myc and AU1 tags. Selected substrates or cleavage products including a 6×-HIS tag can be isolated using metal chelate affinity purification; in the same way, selected substrates or cleavage products with a biotin tag can be isolated using affinity purification with streptavidin; similarly, selected substrates or cleavage products which include FLAG, HA, c-myc, AU1 or other epitope tags can be isolated using antibodies that specifically bind the epitope, for example, commercially available monoclonal antibodies. Detectable tags include, without limitation, fluorescent labels, radiolabels, and enzymes with detectable activity. As non-limiting examples, a detectable tag useful in the invention can be luciferase; an enzyme such as horseradish peroxidase or alkaline phosphatase; or a genetically encoded fluorescent label such as GFP, BFP, YFP or CFP. In one embodiment, protease activity is assayed using a selected substrate which is prepared recombinantly and includes a genetically encoded affinity tag and a genetically encoded detectable tag. In another embodiment, protease activity is assayed using a selected substrate in which the clostridial toxin resistant cleavage site intervenes between GFP and a 6×-HIS tag. An example of a protease assay performed with a GFP-SNAP23-6×HIS substrate is described in Example I.

Assays useful in the invention further include those based on fluorescence resonance energy transfer (FRET), which is a distance-dependent interaction between the electronic excited states of two molecules in which excitation is transferred from a donor fluorophore to an acceptor without emission of a photon. The process of energy transfer results in a reduction (quenching) of fluorescence intensity and excited state lifetime of the donor fluorophore and, where the acceptor is a fluorophore, can produce an increase in the emission intensity of the acceptor. Upon cleavage of a clostridial toxin substrate of the invention, resonance energy transfer is reduced and can be detected, for example, by increased donor fluorescence emission, decreased acceptor fluorescence emission, or by a shift in the emission maxima from near the acceptor emission maxima to near the donor emission maxima. See Clegg, Current Opinion in Biotech. 6:103-110 (1995); and Selvin, Nature Structural Biol. 7:730-734 (2000)). FRET assays useful for detecting protease activity of a clostridial toxin are described, for example, in U.S. Ser. No. 09/942,098 and Anne et al., Analyt. Biochem. 291:253-261 (2001).

Substrates suitable for FRET assays contain a recognition sequence that includes a cleavage site and is resistant to cleavage by naturally occurring clostridial toxins under conditions suitable for clostridial toxin protease activity. Substrates suitable for FRET assays further contain a donor fluorophore and acceptor. Donor fluorophores, also known as fluorochromes, are molecules that, when irradiated with light of a certain wavelength, emit light, also denoted fluorescence, of a different wavelength. Acceptors are molecules that can absorb energy from, and upon excitation of, a donor fluorophore. An acceptor useful the methods of the invention has an absorbance spectrum which overlaps the emission spectrum of the donor fluorophore included in the substrate and generally has rather low absorption at a wavelength suitable for excitation of the donor fluorophore. It is understood that acceptors useful in the methods include both fluorophores as well as non-fluorescent acceptors, sometimes designated a “true quenchers.” A selected substrate useful for assaying for protease activity using FRET contains a cleavage site that intervenes between the donor fluorophore and the acceptor.

A variety of donor fluorophore-acceptor pairs can be useful in a method of the invention. See, for example, Haugland, Handbook of Fluorescent Probes and Research Chemicals 6^{th} Edition, Molecular Probes, Inc., Eugene, Oreg., 1996; Wu and Brand, Analytical Biochem. 218:1-13 (1994); and Berlman, Energy Transfer Parameters of Aromatic Compounds Academic Press, New York 1973). Donor fluorophore/acceptor pairs useful in the invention include, without limitation, fluorescein and tetramethylrhodamine (TMR); fluorescein and QSYC® 7; EDANS and DABCYL; napthalene and dansyl; pairs of Alexa Fluor® dyes; and pairs of genetically encoded dyes such as blue fluorescence protein (BFP) and green fluorescence protein (GFP) or cyan fluorescence protein (CFP) and yellow fluorescence protein (YFP). See Selvin, supra, 2000; Mahajan et al., Chemistry and Biology 6:401-409 (1999); and U.S. Pat. No. 5,981,200.

A selected substrate containing a donor fluorophore and acceptor can be prepared by well known methods (Fairclough and Cantor, Methods Enzymol. 48:347-379 (1978); Glaser et al., Chemical Modification of Proteins Elsevier Biochemical Press, Amsterdam (1975); Haugland, Excited States of Biopolymers (Steiner Ed.) pp. 29-58, Plenum Press, New York (1983); Means and Feeney, Bioconjugate Chem. 1:2-12 (1990); Matthews et al., Methods Enzymol. 208:468-496 (1991); Lundblad, Chemical Reagents for Protein Modification 2nd Ed., CRC Press, Boca Raton, Fla. (1991); Haugland, supra, 1996). A variety of groups can be used to couple a donor fluorophore or acceptor, for example, to a peptide or peptidomimetic substrate. A thiol group, for example, can be used to couple a donor fluorophore or acceptor to the desired position in a peptide or peptidomimetic to produce a substrate useful for assaying for protease activity in a method of the invention. Haloacetyl and maleimide labeling reagents also can be used to couple donor fluorophores or acceptors in preparing a substrate useful in the invention (see, for example, Wu and Brand, supra, 1994).

Methods of performing directed evolution using fluorescence activated cell sorting also can be useful in the invention. Fluorescence activated cell sorting, for example, using a population in which clostridial toxin light chain variants or functional fragments are expressed on E. coli using a FRET peptide substrate can be performed, for example, as described in (Olsen et al., supra, 2000).

Conditions suitable for assaying a population of clostridial toxin light chain variants are the same or similar to those used for assaying for protease activity by wild type toxins and are known in the art or can be determined by routine methods. See, for example, Hallis et al., J. Clin. Microbiol. 34:1934-1938 (1996); Ekong et al., Microbiol. 143:3337-3347 (1997); Shone et al., WO 95/33850; Schmidt and Bostian, supra, 1995; Schmidt and Bostian, J. of Protein Chemistry 16:19-26 (1997); Schmidt et al., FEBS Letters 435:61-64 (1998); and Schmidt and Bostian, U.S. Pat. No. 5,965,699. It is understood that conditions suitable for assaying for protease activity can depend, in part, on the purity of the variants assayed. Conditions suitable for assaying for protease activity towards a selected clostridial toxin-resistant target protein generally include a buffer, such as HEPES, Tris or sodium phosphate, typically in the range of pH 5.5 to 9.5, for example, in the range of pH 6.0 to 9.0, pH 6.5 to 8.5 or pH 7.0 to 8.0. Conditions suitable for assaying for protease activity towards a selected clostridial toxin-resistant target protein also can include, if desired, dithiothreitol, β-mercaptoethanol or another reducing agent, for example, where a dichain toxin is being assayed (Ekong et al., supra, 1997). In one embodiment, the conditions include DTT in the range of 0.01 mM to 50 mM; in other embodiments, the conditions include DTT in the range of 0.1 mM to 20 mM, 1 to 20 mM, or 5 to 10 mM. If desired, dichain toxins including a clostridial toxin light chain variant can be pre-incubated with a reducing agent such as 10 mM dithiothreitol prior to addition of the selected clostridial toxin-resistant target protein.

Clostridial toxins are zinc metalloproteases, and a source of zinc, such as zinc chloride or zinc acetate, typically in the range of 1 to 500 μM, for example, 5 to 10 μM can be included when assaying for protease activity towards a selected clostridial toxin-resistant target protein. One skilled in the art understands that zinc chelators such as EDTA generally are excluded from buffers useful in assaying for protease activity.

Conditions suitable for assaying for protease activity towards a selected clostridial toxin-resistant target protein can optionally include bovine serum albumin (BSA) or detergents such as Tween-20™. When included, BSA typically is provided in the range of 0.1 mg/ml to 10 mg/ml, such as at 1 mg/ml (Schmidt and Bostian, supra, 1997). When included, Tween-20™ typically is provided in the range of 0.01% to 1%, for example, at 0.1%.

The concentration of clostridial toxin light chain variant assayed in a method of the invention generally is in the range of about 0.0001 to 5000 ng/ml light chain variant, for example, about 0.001 to 5000 ng/ml, 0.01 to 5000 ng/ml, 0.1 to 5000 ng/ml, 1 to 5000 ng/ml, or 10 to 5000 ng/ml light chain variant. Generally, the amount of clostridial toxin light chain variant is in the range of 0.1 pg to 1 mg.

Conditions suitable for protease activity towards a selected clostridial toxin-resistant target protein also generally include, for example, temperatures in the range of about 20° C. to about 45° C., for example, in the range of 25° C. to 40° C., or the range of 35° C. to 39° C. Assay volumes often are in the range of about 5 to about 200 μl, for example, in the range of about 10 μl to 100 μl or about 0.5 μl to 100 μl, although nanoliter reaction volumes also can be useful the methods of the invention. Assay volumes also can be, for example, in the range of 100 μl to 2.0 ml or in the range of 0.5 ml to 1.0 ml.

Assay times can be varied as appropriate by the skilled artisan and generally depend, in part, on the concentration and purity and activity of the clostridial toxin light chain variant. Protease reactions can be terminated, for example, by addition of H₂SO₄, addition of about 0.5 to 1.0 M sodium borate, pH 9.0 to 9.5; addition of zinc chelators; or addition of guanidine chloride to a final concentration of about 1 to 2 M. One skilled in the art understands that, where FRET assays are used, the protease reactions can be terminated, if desired, prior to exciting the donor fluorophore or determining energy transfer.

As an example, conditions suitable for assaying clostridial toxin light chain variants, for example, variants of BoNT/A are incubation at 37° C. in a buffer such as 30 mM HEPES (pH 7.3); a source of zinc such as 25 μM zinc chloride; and about 1 μg/ml light chain variant (Schmidt and Bostian, supra, 1997). BSA in the range of 0.1 mg/ml to 10 mg/ml, for example, 1 mg/ml BSA, also can be included. As another example, conditions suitable for assaying for protease activity towards a selected clostridial toxin-resistant target protein can be incubation at 37° C. for 30 minutes in a buffer containing 50 mM HEPES (pH 7.4), 1% fetal bovine serum, 10 μM ZnCl₂ and 10 mM DTT with 10 μM substrate; incubation in 50 mM HEPES, pH 7.4, with 10 μM zinc chloride, 1% fetal bovine serum and 10 mM dithiothreitol, with incubation for 90 minutes at 37° C. (Shone and Roberts, Eur. J. Biochem. 225:263-270 (1994); Hallis et al., supra, 1996); or can be, for example, incubation in 40 mM sodium phosphate, pH 7.4, with 10 mM dithiothreitol, optionally including 0.2% (v/v) Triton X-100, with incubation for 2 hours at 37° C. (Shone et al., supra, 1993). Conditions suitable for assaying for tetanus toxin light chain variants or other light chain variants can be, for example, incubation in 20 mM HEPES, pH 7.2, and 100 mM NaCl for 2 hours at 37° C. with 25 μM peptide substrate (Cornille et al., Eur. J. Biochem. 222:173-181 (1994)).

It is understood that the methods of the invention can be automated and can be configured in a high-throughput or ultra high-throughput format using, for example, 96-well, 384-well or 1536-well plates. Where assays are fluorescence-based, fluorescence emission can be detected, for example, using Molecular Devices FLIPR® instrumentation system (Molecular Devices; Sunnyvale, Calif.), which is designed for 96-well plate assays (Schroeder et al., J. Biomol. Screening 1:75-80 (1996)). One skilled in the art understands that a variety of other automated systems can be useful in the methods of the invention.

A method of the invention optionally includes digital imaging to assay for protease activity towards the selected clostridial toxin-resistant target protein. As an example, digital imaging as a function of time can be used to obtain an estimate of the Vₘₐₓ of variants expressed by individual colonies. Digital analysis can be performed rapidly with, for example, about 10⁵ colonies assayed per day using an advanced digital imaging system (Joo et al., Chem. Biol. 6:699-706 (1999); Joo et al., Nature 399:670-673 (1999); and Bylina et al., ASM News 66:211-217 (2000)).

### EXAMPLE I

### Fluorescence Release Assay for Botulinum Neurotoxin Protease Assay

This example describes a fluorescence release assay using a GFP-SNAP23 substrate.

Plasmids encoding the selected substrates are prepared by modifying vector PQBI T7-GFP (Qbiogene, Inc.; Carlsbad, Calif.) as described below. Plasmid pQBI GFP-SNAP23 is constructed in two phases. First, vector PQBI T7-GFP is PCR-modified to remove the stop codon at the 3′ terminus of the GFP-coding sequence and to insert the coding sequence for a portion of the peptide linker separating GFP from a SNAP-23 fragment. Second, a DNA fragment coding for SNAP-23 is PCR amplified. The PCR primers are designed to incorporate the coding sequence for the remainder of the peptide linker fused 5′ to the SNAP-23 sequence and a 6×-HIS affinity tag fused 3′ of the gene. The resultant PCR product is cloned into the modified pQBI vector to yield the desired pQBI GFP-SNAP23 plasmid for expression of GFP-linker-SNAP-23-6×-HIS.

Plasmid pQBI SNAP23-GFP is constructed by subcloning a PCR amplified gene containing the BirAsp biotinylation sequence, a poly-His affinity tag, and the appropriate portion of SNAP-23 into PQBI T7-GFP using PCR amplification. The PCR primers are designed to incorporate the coding sequence for a fusion protein linker 3′ of the amplified gene and to facilitate fusion to the 5′ terminus of the GFP gene, yielding a single gene for expression of BirAsp-6×HIS-SNAP23-linker-GFP.

Expression of fusion proteins is performed as follows. Plasmid pQBI GFP-SNAP23 is transformed into E. coli BL21-CodonPlus® (DE3)-RIL cells (Stratagene; La Jolla, Calif.) containing the T7 RNA polymerase gene. The transformed cells are spread onto LB plates containing ampicillin and incubated overnight at 37° C. Single colonies are used to inoculate 3 ml overnight cultures, which are in turn used to inoculate four 500 ml cultures. The cultures are grown at 37° C. with shaking until A₅₉₅ reaches 0.5-0.6, at which time they are removed from the incubator and allowed to cool. Protein expression is induced by addition of 1 mM IPTG, and the cultures incubated overnight at 25° C. with shaking. The protein is expressed below 30° C. such that the GFP fluorophore forms properly. Cells from a 250 ml culture are pelleted and stored at −80° C. until further use.

Fusion proteins are purified essentially as follows with all steps performed at 4° C. Briefly, the cell pellet from a 250 ml culture is resuspended in 12 to 15 ml Column Binding Buffer (25 mM HEPES, pH 8.0; 500 mM NaCl; 1 mM β-mercaptoethanol; and 10 mM imidazole), lysed by sonication (140 sec in 10-sec pulses at 38% amplitude), and clarified by centrifugation (16,000 rpm, 4° C., 1 hour). Affinity resin (10 ml Talon SuperFlow Co²⁺) is equilibrated in a 20 ml column support (Bio-Rad; Hercules, Calif.) by rinsing with 8 column volumes of distilled water and 8 column volumes of Column Binding Buffer. The clarified lysate is added to the resin and batch bound by horizontal incubation for 1 hour with gentle rocking. Following batch binding, the column is righted and the solution is drained, collected, and passed over the resin again. The column is then washed with 8 column volumes of Column Wash Buffer (25 mM HEPES, pH8.0; 500 mM NaCl; 1 mM β-mercaptoethanol; 20 mM imidazole), and the protein eluted with 15 ml Column Elution Buffer (25 mM HEPES, pH 8.0; 500 mM NaCl; 1 mM β-mercaptoethanol; 250 mM imidazole), which is collected in fractions of ˜1.4 mL. The green fractions are combined and desalted by FPLC (BioRad Biologic DuoLogic, QuadTec UV-Vis detector) with a HiPrep 26/10 size exclusion column (Pharmacia) and an isocratic mobile phase of chilled Fusion Protein Desalting Buffer (50 mM HEPES, pH 7.4, 4° C.) at a flow rate of 10 ml/min. The desalted protein is collected as a single fraction, concentrated in an Apollo 20-ml concentrator (QMWL 10 kDa, Orbital Biosciences), and the concentration determined by the BioRad Protein Assay. The protein solution is divided into 500 ml aliquots, flash-frozen with liquid nitrogen and stored at −80° C. Once defrosted, a working aliquot is stored at 4° C., protected from light.

SDS-PAGE and Western blot analysis of proteolysis reactions is performed as follows. GFP-SNAP23-6×HIS substrate (0.4 mg/mL) is combined with a population of clostridial toxin light chain variants or functional fragments in toxin reaction buffer (50 mM HEPES, pH 7.4, 0.1% (v/v) Tween-20, 10 μM ZnCl₂, 10 mM DTT). The reactions are incubated at 37° C., with aliquots removed after incubation for 0, 5, 10, 15, 30, and 60 minutes and quenched by addition to gel loading buffer. Reactions mixtures are analyzed by SDS-PAGE (10% Bis-Tris MQPS) and staining with Sypro Ruby (Bio-Rad; Hercules, Calif.) or are transferred to nitrocellulose membranes and probed with antibodies specific for GFP or SNAP-23 cleavage product.

The proteolysis assay is performed either using spin-column processing or filter-plate processing, as described further below. The GFP substrates are handled under low-light conditions to reduce photobleaching.

Spin Column Processing

The assay using spin column processing is performed as follows. Spin columns and filters (35 μM pore size; MoBiTec; Guettingen, Germany) are assembled and loaded with 100 μl of Talon™ Superflow Co²⁺ affinity resin (BD Biosciences; San Jose, Calif.). Columns are fitted with a Luer-lock cap, and the resin storage buffer eluted by syringe pressure. Resin is conditioned by rinsing with 1 ml dH₂O and 1 ml Assay Rinse Buffer (50 mM HEPES, pH 7.4).

The clostridial toxin light chain variants or functional fragments are diluted to twice the desired reaction concentration with Toxin Reaction Buffer (50 mM HEPES, pH 7.4; 10 μM ZnCl₂; 10 mM DTT; and 0.1% (v/v) Tween-20) and are added to black v-bottom 96-well plates (Whatman) in 50 μl aliquots. Each clostridial toxin light chain variant or functional fragment is pre-incubated at 37° C. for 20 minutes, at which time the reaction is initiated by addition of substrate. Prior to initiation of the reactions, the GFP-SNAP substrate is diluted with Toxin Reaction Buffer to a 2× working concentration and is warmed to 37° C. Reactions are initiated by addition of 50 μl substrate to yield a final reaction volume of 100 μl (16 mM GFP-SNAP). The reaction plates are covered with film, protected from light, and incubated at 37° C. for 1.5 hours. All reactions are run in triplicate. Following the desired reaction time, reactions are quenched by addition of 8 M guanidine hydrochloride (15 μl) and are transferred to columns containing conditioned Co²⁺ resin. Samples are incubated with resin for 15 minutes at room temperature; the columns are then eluted by centrifugation at 2000 rpm for 30 seconds into 1.7 ml microcentrifuge tubes. Eluant or reaction flow-through (containing GFP product) is then passed over the columns two additional times and saved after the final pass. Each column is then rinsed with 140 μl Assay Rinse Buffer, and the flow-through is collected into the same tube as the reaction flow-through. Columns are then washed twice with 250 μl and once with 350 μl Assay Rinse Buffer. Unreacted substrate is eluted from the column with 250 μl Assay Elution Buffer (50 mM HEPES, pH 7.4; 250 mM imidazole). The eluant solutions corresponding to the reaction flow-through and imidazole eluant solutions are transferred to a black, flat-bottom 96-well microtiter plate (Whatman; Kent, United Kingdom), and the fluorescence quantified with a SpectraMax Gemini XS spectrophotometer (Molecular Devices, λEx 474 nm; λEm 509 nm; 495 nm cutoff filter).

Filter-Plate Processing

For filter-plate processing, the required number of wells in a 96-well filter plate (400 μl wells, 0.45 μm filter, long drip; Innovative Microplate; Chicopee, Mass.) are loaded with 75 μl of Talon™ Superflow Co²⁺ affinity resin (BD Biosciences; San Jose, Calif.). Unused wells are sealed with tape, and the plate placed in a UniVac® vacuum manifold (Whatman) for elution at 10 to 20 inches mercury. Resin storage buffer is removed by vacuum, and the resin conditioned by rinsing twice with 250 μl distilled water and twice with 250 μl Assay Rinse Buffer (50 mM HEPES, pH 7.4). The last aliquot of Assay Rinse Buffer is eluted immediately prior to transfer of reaction solutions to the filter plate.

Clostridial toxin light chain variants or functional fragments are diluted to twice the desired reaction concentration with Toxin Reaction Buffer (50 mM HEPES, pH 7.4; 10 μM ZnCl₂; 10 mM DTT; and 0.1% (v/v) Tween-20) and added to black v-bottom 96-well plates (Whatman) in 25 μl aliquots. Each clostridial toxin light chain variant or functional fragment is pre-incubated at 37° C. for 20 minutes, when the reaction is initiated by addition of substrate. Prior to initiation of the reactions, the GFP-SNAP substrate is diluted with Toxin Reaction Buffer to a 2× working concentration and is warmed to 37° C. Reactions are initiated by addition of 25 μl substrate to yield a final reaction volume of 50 μl (13.5 μM GFP-SNAP). The reaction plates are covered with film, protected from light, and incubated at 37° C. for one hour. All reactions are run in triplicate. Following the desired reaction time, reactions are quenched by the addition of 8 M guanidine hydrochloride (15 μL) and transferred to filter plate wells containing conditioned Co²⁺ resin, where they are incubated at room temperature for 15 minutes. The reaction solutions are eluted, collected in a black, flat-bottom 96-well plate, passed over the resin beds twice more and collected after the final pass. Each resin bed is then rinsed with 200 μl Assay Rinse Buffer which is eluted into the plate containing the eluant reaction solution, or reaction flow-through (containing GFP product). The resin beds are then washed three times with 250 μL Assay Rinse Buffer. Unreacted substrate is eluted from the resin beds with 250 μl Assay Elution Buffer 500 (50 mM HEPES, pH 7.4; 500 mM imidazole) and collected in a black, flat-bottom 96-well plate. The fluorescence of the reaction flow-through and imidazole eluant solutions is quantified with a SpectraMax Gemini XS spectrophotometer (Molecular Devices, λ_{Ex} 474 nm; λ_{Em} 509 nm; 495 nm cutoff filter).

This example demonstrates that fluorescent substrates containing an affinity tag can be used to assay for clostridial toxin protease assay.

All journal article, reference and patent citations provided above, in parentheses or otherwise, whether previously stated or not, are incorporated herein by reference in their entirety.

Although the invention has been described with reference to the examples provided above, it should be understood that various modifications can be made without departing from the spirit of the invention. Accordingly, the invention is limited only by the following claims.

We claim:

1. A method of producing an evolved clostridial toxin light chain having altered protease specificity, comprising the steps of:
(a) generating a population, each member of said population comprising a clostridial toxin light chain variant, or functional fragment thereof;
(b) assaying said population for protease activity towards a selected clostridial toxin-resistant target protein, wherein increased protease activity is indicative of an evolved clostridial toxin light chain; and
(c) isolating from said population one or more members comprising an evolved clostridial toxin light chain or functional fragment thereof.

2. The method of claim 1, wherein said altered protease specificity is for a clostridial toxin-resistant SNARE protein.

3. The method of claim 2, wherein said clostridial toxin-resistant SNARE protein is human SNAP-23.

4. The method of claim 2, wherein said clostridial toxin-resistant SNARE protein is syncollin.

5. The method of claim 2, wherein said clostridial toxin-resistant SNARE protein is TI-VAMP.

6. The method of claim 1, wherein said population is a random population.

7. The method of claim 1, wherein step (a) comprises expressing a population of nucleic acid molecules encoding a population of clostridial toxin light chain variants or functional fragments thereof.

8. The method of claim 7, comprising genetic modification of one or more nucleic acid molecules encoding a clostridial toxin light chain or segment thereof.

9. The method of claim 8, wherein said genetic modification is random mutagenesis.

10. The method of claim 9, wherein said population comprises at least 10² different members, each member comprising a clostridial toxin light chain variant or functional fragment thereof.

11. The method of claim 9, wherein said population comprises at least 10³ different members, each member comprising a clostridial toxin light chain variant or functional fragment thereof.

12. The method of claim 9, wherein said random mutagenesis yields an average of 1 to 3 amino acid substitutions per clostridial toxin light chain variant or functional fragment thereof.

13. The method of claim 9, wherein said random mutagenesis comprises error-prone polymerase chain reaction amplification.

14. The method of claim 9, wherein said random mutagenesis comprises DNA shuffling between two or more nucleic acid molecules encoding clostridial toxin light chains or segments thereof.

15. The method of claim 9, wherein said random mutagenesis comprises saturation mutagenesis of one or more codons of said one or more nucleic acid molecules or segments thereof.

16. The method of claim 9, wherein step (b) comprises assaying a population of microorganisms, each microorganism expressing a clostridial toxin light chain variant or functional fragment thereof.

17. The method of claim 16, wherein said clostridial toxin light chain variant or functional fragment thereof is expressed on the cell surface of said microorganism.

18. The method of claim 16 or claim 17, wherein said microorganism is Escherichia coli.

19. The method of claim 9, wherein step (b) comprises assaying a population of phage, each phage expressing a clostridial toxin light chain variant or functional fragment thereof.

20. The method of one of claims 16 through 19, wherein step (b) comprises selection of one or more viable members from said population, each viable member comprising an evolved clostridial toxin light chain or functional fragment thereof.

21. The method of claim 9, wherein step (b) comprises assaying a population of purified or partially purified polypeptides or functional fragments thereof.

22. The method of claim 21, wherein said population is a population of purified clostridial toxin light chain variants or functional fragments thereof.

23. The method of claim 21, wherein said population is a population of purified toxins, each toxin comprising a clostridial toxin heavy chain and a clostridial toxin light chain variant.

24. The method of claim 23, wherein said population is a population of dichain toxins.

25. The method of claim 9, wherein step (b) comprises an immunoassay.

26. The method of claim 25, wherein said immunoassay is an enzyme-linked immunosorbent assay (ELISA).

27. The method of claim 9, wherein step (b) comprises a fluorescence resonance energy transfer (FRET) assay.

28. The method of claim 1, 16 or 17, wherein step (b) comprises fluorescence activated cell sorting (FACS).

29. The method of claim 1, wherein steps (a), (b) and (c) are repeated one or more times.

30. The method of claim 29, wherein steps (a), (b) and (c) are repeated three or more times.

31. The method of claim 1, wherein said clostridial toxin light chain variants are botulinum toxin light chain variants.

32. A composition, comprising an evolved clostridial toxin light chain or functional fragment thereof having altered protease specificity.

33. The composition of claim 32, wherein said altered protease specificity is for a clostridial toxin-resistant SNARE protein.

34. The composition of claim 33, wherein said clostridial toxin-resistant SNARE protein is human SNAP-23.

35. The composition of claim 32, wherein, under the appropriate conditions, said altered protease specificity inhibits exocytosis.

36. The composition of claim 35, wherein, under the appropriate conditions, said altered protease specificity inhibits neuronal exocytosis.

37. The composition of claim 35, wherein, under the appropriate conditions, said altered protease specificity inhibits secretory cell exocytosis.

38. The composition of claim 37, wherein said secretory cell exocytosis is pancreatic acinar cell exocytosis.

39. The composition of claim 32, which differs from a naturally occurring clostridial toxin light chain by at most three amino acid substitutions.

40. The composition of claim 32, which differs from a naturally occurring clostridial toxin light chain by a single amino acid substitution.

41. The composition of claim 32, further comprising a clostridial toxin heavy chain.

42. The composition of claim 41, wherein said clostridial toxin heavy chain has a non-naturally occurring amino acid sequence.

43. The composition of claim 42, wherein said clostridial toxin heavy chain has a non-naturally occurring binding domain.

44. A nucleic acid molecule, comprising a nucleic acid sequence encoding an evolved clostridial toxin light chain having altered protease specificity, or a functional fragment thereof.

45. The nucleic acid molecule of claim 44, wherein said altered protease specificity is for a clostridial toxin-resistant SNARE protein.

46. The nucleic acid molecule of claim 45, wherein said clostridial toxin-resistant SNARE protein is human SNAP-23.

47. The nucleic acid molecule of claim 44, wherein, under the appropriate conditions, said altered protease specificity inhibits exocytosis.

48. The nucleic acid molecule of claim 47, wherein, under the appropriate conditions, said altered protease specificity inhibits neuronal exocytosis.

49. The nucleic acid molecule of claim 47, wherein, under the appropriate conditions, said altered protease specificity inhibits secretory cell exocytosis.

50. The nucleic acid molecule of claim 49, wherein said secretory cell exocytosis is pancreatic acinar cell exocytosis.

51. The nucleic acid molecule of claim 44, wherein said evolved clostridial toxin light chain differs from a naturally occurring clostridial toxin light chain by at most three amino acid substitutions.

52. The nucleic acid molecule of claim 44, wherein said evolved clostridial toxin light chain differs from a naturally occurring clostridial toxin light chain by a single amino acid substitution.

53. The nucleic acid molecule of claim 44, further comprising a nucleic acid sequence encoding a clostridial toxin heavy chain.


SEQUENCE LISTING (12 sequences)

SEQ ID NO: 1 - protein, 211 aa, Homo sapiens
MDNLSSEEIQQRAHQITDESLESTRRILGLAIESQDAGIKTITMLDEQKEQLNRIEEGLDQINKDMRETEKTLTELNKCCGLCVCPCNRTKNFESGKAYKTTWGDGGENSPCNVVSKQPGPVTNGQLQQPTTGAASGGYIKRITNDAREDEMEENLTQVGSILGNLKDMALNIGNEIDAQNPQIKRITDKADTNRDRIDIANARAKKLIDS

SEQ ID NO: 2 - protein, 158 aa, Homo sapiens
MDNLSSEEIQQRAHQITDESLESTRRILGLAIESQDAGIKTITMLDEQKEQLNRIEEGLDQINKDMRETEKTLTELNKCCGLCVCPCNSITNDAREDEMEENLTQVGSILGNLKDMALNIGNEIDAQNPQIKRITDKADTNRDRIDIANARAKKLIDS

SEQ ID NO: 3 - protein, 206 aa, Homo sapiens
MAEDADMRNELEEMQRRADQLADESLESTRRMLQLVEESKDAGIRTLVMLDEQGEQLDRVEEGMNHINQDMKEAEKNLKDLGKCCGLFICPCNKLKSSDAYKKAWGNNQDGVVASQPARVVDEREQMAISGGFIRRVTNDARENEMDENLEQVSGIIGNLRHMALDMGNEIDTQNRQIDRIMEKADSNKTRIDEANQRATKMLGSG

SEQ ID NO: 4 - protein, 8 aa, Homo sapiens
EANQRATK

SEQ ID NO: 5 - protein, 8 aa, Homo sapiens
GASQFETS

SEQ ID NO: 6 - protein, 8 aa, Homo sapiens
DTKKAVKY

SEQ ID NO: 7 - protein, 8 aa, Homo sapiens
RDQKLSEL

SEQ ID NO: 8 - protein, 8 aa, Homo sapiens
QIDRIMEK

SEQ ID NO: 9 - protein, 8 aa, Homo sapiens
ERDQKLSE

SEQ ID NO: 10 - protein, 8 aa, Homo sapiens
ETSAAKLK

SEQ ID NO: 11 - protein, 8 aa, Homo sapiens
GASQFETS

SEQ ID NO: 12 - protein, 10 aa, Artificial Sequence
  synthetic construct
EIDAQNPQIK